(19) United States
(12) Patent Application Publication
(10) Pub. No.: US 2013/0209585 A1
(43) Pub. Date: Aug. 15, 2013

(54) Topical NMDA Antagonist Formulations for the Treatment of Peripheral Neuropathy

(71) Applicant: Stanley Kim, San Diego, CA (US)

(72) Inventor: Stanley Kim, San Diego, CA (US)

(21) Appl. No.: 13/766,456

(22) Filed: Feb. 13, 2013

Related U.S. Application Data
(60) Provisional application No. 61/598,169, filed on Feb. 13, 2012.

(51) Int. Cl. A61K 47/44

(52) U.S. Cl. CPC A61K 47/44
USPC 424/722; 514/647

(57) ABSTRACT

The presenting invention is directed to methods and compositions for the topical or transdermal treatment of neuropathy. More particularly, transdermal or topical compositions including a combination of ingredients that provide a surprising degree of effective relief from the symptoms of peripheral neuropathy and methods for administering the compositions to treat various neuropathies.

### CROSS REFERENCE TO RELATED APPLICATIONS

This application claims the benefit under 35 U.S.C. §119(e) to U.S. Provisional Application Ser. No. 61/598,169 filed Feb. 13, 2012.

### FIELD OF THE INVENTION

The present invention relates to methods and compositions for the topical treatment of neuropathy. More particularly, the present invention relates to topical compositions including the local delivery of an NMDA antagonist in a topical vehicle that limits NMDA antagonist penetration into the blood while providing effective relief from the symptoms of neuropathy, and to methods for administering topical compositions to treat neuropathy.

### BACKGROUND

Peripheral neuropathy is a condition involving nerve-end damage anywhere in the body. Peripheral neuropathy generally refers to a disorder that affects the peripheral nerves, most often manifested as one or a combination of motor, sensory, sensorimotor, or autonomic neural dysfunction. The wide variety of morphologies exhibited by peripheral neuropathies can each be uniquely attributed to an equally wide variety of causes. For instance, peripheral neuropathies can be genetically acquired, can result from a systemic disease, can manifest as a post-surgical complication, or can be induced by a toxic agent. Some toxic agents that cause neurotoxicities are therapeutic drugs, antineoplastic agents, contaminants in foods or medicinals, and environmental and industrial pollutants. As much as 3% of the population is estimated to be affected, if not greater.

Although a number of neuropathies are related to the disease diabetes mellitus, others, although not known to be related to diabetes are similar in their physiological effects on the peripheral vascular system. Such diseases include Raynaud's Phenomenon, including CREST syndrome, autoimmune diseases such as erythromatosis, and rheumatoid diseases. Other peripheral neuropathies include the following: HIV-associated neuropathy; nutritional deficiency-associated neuropathy; cranial nerve palsies; drug-induced neuropathy; industrial neuropathy; lymphomatous neuropathy; myelomatous neuropathy; multi-focal motor neuropathy; immune-mediated disorders, chronic idiopathic sensory neuropathy; carcinomatous neuropathy; acute pain autonomic neuropathy; alcoholic neuropathy; compressive neuropathy; vasculitic/ischaemic neuropathy; mono- and polyneuropathies.

For example, among the most important toxic agents causing peripheral neuropathy are therapeutic agents, particularly those used for the treatment of neoplastic disease. In certain cases, peripheral neuropathy is a major complication of cancer treatment and is the main factor limiting the dosage of chemotherapeutic agents that can be administered to a patient (Macdonald, Neurologic Clinics 9:955-967 (1991)). This is true for the commonly administered agents cisplatin, paclitaxel and vincristine (Broun, et al., Am. J. Clin. Oncol. 16:18-21 (1993); Macdonald, Neurologic Clinics 9:955-967 (1991); Casey, et al., Brain 96:69-86 (1973)). The identification of methods for preventing or alleviating dose-limiting peripheral neuropathologic side effects would allow higher, and more therapeutically effective doses of these chemotherapeutics to be administered to patients, i.e., the therapeutic efficacy of such chemotherapeutics is typically a function of dose and therefore, increasing dosage provides increased patient survival (Macdonald, Neurologic Clinics 9:955-967 (1991); Oxols, Seminars in Oncology 16, suppl. 6:22-30 (1989))

Tragically there is no existing method for adequately, predictably and specifically treating established neuropathic pain (Woolf C. et al., Neuropathic Pain: Aetiology, Symptoms, Mechanisms, and Management, Lancet 1999; 353: 1959-64). Present treatment methods for neuropathic pain consists of merely trying to help the patient cope through psychological or occupational therapy, rather than by reducing or eliminating the pain experienced.

N-methyl-D-aspartate (NMDA) receptors are one of the major excitatory neurotransmitter receptors in the brain and spinal cord major excitatory system. There is accumulating evidence to implicate the importance of NMDA receptors to the induction and maintenance of central sensitization during pain states. NMDA receptors may also mediate peripheral sensitization and visceral pain. (Cairns B E et al., 2003, J Neurophysiol 90:2098-105; Petrenko A B, et al., 2003 Anesth Analg 97:1108-1116).

Unfortunately, only a few NMDA antagonists are clinically available and their use is limited by unacceptable side effects. Generally, depressed NMDA receptor function is associated with an array of negative symptoms. They sometimes induce “psychotomimetic” side effects, symptoms resembling psychosis. Such side effects caused by NMDA receptor inhibitors can include hallucinations, paranoid delusions, confusion, dizziness, tachycardia, increased blood pressure, disorientation, delirium, difficulty concentrating or light-headedness, agitation, convulsions, alterations in mood and personality, nightmares (Muir, K W; Lees K R, 1995 Stroke 26(3):503-513.), catatonia (Aarts, M M; Tymianski M., 2003, Biochemical Pharmacology 66 (6): 877-886.), ataxia (Kim A H, Kerchner G A, and Choi D W. (2002). “Blocking Excitotoxicity”. In CNS Neuroprotection. Marcoux F W and Choi D W, editors. Springer, New York. Pages 3-36), anaesthesia (Kristensen, J D; Svensson B, and Gordh T Jr., 1992, Pain 51 (2): 249-253) and learning and memory deficits (Rockstroh, S; Emre M, Tarral A, and Pokorny R., 1996, Psychopharmacology 124(3):261-266.) In certain animals, such as rats, certain NMDA antagonists cause neurotoxicity and permanent brain injury (see, e.g., Olney J, Labruyerre J and Price M T. 1989. Science, 244(4910):1360-1362; Ellison G. 1995. Brain Research. Brain Research Reviews, 20(2):250-267).

Ketamine ((2-(2-chlorophenyl)-2-(methylamino)-cyclohexanone) is a general anesthetic used by anesthesiologists, veterinarians, and researchers. Ketamine is an N-methyl-D-aspartate receptor antagonist and thus blocks a cascade of intracellular events that inhibit the hyper excitability of spinal cord neurons. Usually, ketamine is administered intramuscularly (i.m.) or intravenously (i.v.) for induction of anesthesia. Ketamine has also been known to have analgesic properties (Domino et al., 1965, Clin. Pharmacol. Ther. 6:279); particularly at subanesthetic doses of ketamine (Bovill, 1971, Br. J. Anaesth. 43:496; Sadove et al., 1 971, Anesth. Analg. 50:452-457; Padilla et al., 2000, J Am Dent Assoc, 131:184-195; Bell R. Pain 2009; 141:210-4). Animal data show that certain spontaneous pains and allodynia have been treated successfully with Ketamine.

In humans, several randomised, double-blind, placebo-controlled studies have reported on the reduction of allodynia following intravenous administration of ketamine (Chizh B, Headley P. Curr Pharm Des 2005; 23:2977-94; Eide P, et al. Pain 1994; 58:347-54; Eide P, Stubhaug A, Stenehjem A. Neurosurgery 1995; 37:1080-7; Felsby S, et al. Pain 1995; 64:283-91; Max M, et al. Clin Neuropharmacol 1995; 18:360-8). Trials of the use of ketamine in the treatment of neuropathic pain states have largely revolved around its intravenous administration although it is also administered by various routes, including intramuscular., caudal, epidural, intrathecal, and subcutaneous (Arendt-Nielsen L, et al. Anesth Analg 1996; 81:63-8; Backonja M, et al. Pain 1994; 56:51-7; Bovill J, Dundee J. Br J Anaesth 1971; 43:496-9; Correll G, et al. Pain Med 2004; 5:263-75; Eide P, et al. Pain 1994; 58:347-54; Eide P, Stubhaug A. Neurosurgery 1997; 41:505-8; Eide P, et al. Pain 1995; 61:221-8; Felsby S, et al. Pain 1995; 64:283-91; Goldberg M, et al. Pain Physician 2005; 8:175-9; Grant I, et al. Br J Anaesth 1981; 53:805-10; Harbut R, Correll G. Pain Med 2002; 3:147-55. Hocking G, Cousins M. Anesth Analg 2003; 97:1730-9; Kiefer R-T, et al. Pain Pract 2007; 7:147-50; Kiefer R-T, et al. Pain Med 2007; 9:1173-201; Kiefer R-T, et al. Pain Med 2008; 9:44-54; Koffler S, et al. Arch Clin Neuropsychol 2007; 22:719-29; Mercadante S, et al. J Pain Symptom Manage 1995; 10:564-8. Nikolajsen L, et al. Pain 1996; 67:69-77; Oshima E, et al. Can J Anaesth 1990; 37:385-6; Persson J, et al. Pain 1995; 60:217-22; Shirani P, et al. Pain Physician 2008; 11:339-42; Stannard C, et al. Pain 1993; 54:227-30; Takahashi H, et al. Pain 1998; 75:391-4; Warncke T, et al. Pain 1997; 72:99-106; Yang C-Y, et al. Can J Anaesth 1996; 43:379-83). Subcutaneous administration of ketamine has been used to treat pain following surgery and associated with terminal cancer (see, e.g., Oshima et al., 1990, Can. J. Anaesth. 37:385-386). Ketamine hydrochloride administered via a subcutaneous cannula was reported to successfully treat phantom limb pain (Stannard and Porter, 1993, Pain 54:227-230).

Topical ketamine has been shown to reduce pain for patients with postherpetic neuralgia and other peripheral neuropathies with minimal systemic side effects, particularly at lower dosages to minimize any side effects (Crowley K, et al. Int J Pharm Compd 1998; 2:122-7; Gammaitoni A, et al. Pain Med 2000; 1:97-100; Ho K-Y, et al. Clin J Pain 2008; 24:51-5; Kronenberg R. J Pain Palliat Care Pharmacother 2002; 16:27-35; Lynch M, et al. Anesthesiology 2005; 103:140-6; Lynch M, et al. J Pain 2005; 6:644-9; Pöyhiä R, Vainio A. Clin J Pain 2006; 22:32-6; Quan D, et al. Neurology 2003; 60:1391-2; Slatkin N, Rhiner M. Pain Med 2003; 4:298-303; Ushida T, et al. Reg Anesth Pain Med 2002; 27:524-8; Jones, M., 2002, Int. J. Pharm. Comp. 6(1):4-6; Zapantis et al., 2006, Acta Biologica Hungarica, 57(3):387-389; FitzGibbon E J et al., 2002 J Pain Symptom Manage 23: 165-70). The use of ketamine topically in an organogel has shown some promise in the treatment of neuropathy (Gammaitoni A, Gallagher R M, Welz-Bosna M. Pain Med. 2000 March; 1(1):97-100; Finch P M, Knudsen L, Drummond P D Pain. 2009 November; 146(1-2):18-25). Also, in humans, phantom limb pain has been treated with success (Nadine & Bouhassira, Acta. Neurol. Scand 1999 (Supp 173):12-24). The topical form of Ketamine is effective in treating painful neuropathy when other traditional medicines have failed. (Crowley et al., International Journal of Pharmaceutical Compounding 1998; 2:122-1273). Although this medication has promise for the treatment of neuropathies, the concern for adverse effects such as hallucinations and dysphoria necessitated a low dose (Warncke et al., 1997, Pain, 72(1-2):99-106; Felsby et al., 1996, Pain, 64:283-91).

It has assumed that the mechanism of action for peripheral pain reduction would be the reduction of central sensitization caused by the absorption of topical ketamine in circulation (Pöyhiä, R, Vainio A, 2006. Clin J Pain. 22(1):32-36). Such activity, which may work to alleviate the central sensitization also contribute to the side effect profile of NMDA antagonists. Notwithstanding this, NMDARs have also been identified on unmyelinated and myelinated axons in peripheral somatic tissues (Carlton S M, et al., 1995, Neurosci Lett 197:25-8. Coggeshall R E, Carlton S M. 1998, J Comp Neurol 391:78-86), and are expressed on nerves in human tendons (Alfredson H, et al., J Orthop Res 2001; 19: 881-6). The present invention provides evidence that NMDA antagonists may work at a local level topically at the site of injury, or adjoining or connecting neurotomes.

Accordingly, there remains a need in the art for effective treatments for neuropathies, and other neuropathic pains, particularly for treatments that may act at or near the site of pain that minimize the potential for side effects.

### SUMMARY OF THE INVENTION

In one aspect, the compositions described herein can provide for the treatment of peripheral neuropathy, and can include a therapeutically effective amount of NMDA antagonist in a topical vehicle that limits penetration of the NMDA antagonist generally to the skin, particularly to the epidermis, dermis and the dermatomes. In preferred embodiments, the NMDA is in high concentrations, e.g., generally considered by those skilled in the art as high, such as in itself could lead to psychomimetic symptoms in a representative sample, e.g., 1 or more. In a more preferred embodiment, the present invention is ketamine or norketamine in concentrations equal or greater than 15% volume, more preferably equal or greater than 20%.

The topical preparations described herein include any formulations suitable for topical application, and include: aqueous creams, ointments, gels, lotions, roll-on liquids, sprays, glass bead wound dressings, and synthetic polymer dressings impregnated with the compositions described herein. These preparations may also include compounds that would facilitate the passage of the active ingredients across the skin keratin barrier and into the skin. Preferably the preparation is a cream or ointment. Other formulations the compositions can be incorporated into include oils, suppositories, foams, liniments, aerosols, buccals, and sublingual tablets or topical devices for absorption through the skin or mucous membranes. An additional ingredient can be added as needed to increase the analgesic effectiveness of the combination.

In other aspects, methods described herein are directed to treating peripheral neuropathy, comprising the step of transdermal or topical administration of an effective amount of a pharmaceutical composition comprising an NMDA antagonist in a topical or transdermal vehicle to the affected area of a subject in need of such treatment. Other drugs or ingredients may be added as needed to increase the analgesic effect or minimize the side effects.

In preferred embodiments, the peripheral neuropathy is a diabetic neuropathy. It will be clearly understood that the diabetic neuropathy may be associated with Type 1 (insulin-dependent) diabetes, Type 2 (non-insulin-dependent) diabetes, or both.

In other preferred embodiments, the neuropathy is a non-diabetic neuropathy. Such a non-diabetic neuropathy may be genetically acquired, such as Charcot-Marie-Tooth syndrome. In other embodiments the peripheral neuropathy can result from a systemic or infectious disease such as HIV, or an infectious disease condition such as AIDS. In further embodiments, the peripheral neuropathy manifests as a post surgical complication.

In other embodiments the peripheral neuropathy is induced by a toxic agent. For example, the peripheral neuropathy can be caused by a chemotherapeutic agent such as paclitaxel (or other taxane derivative), vincristine, cisplatin, an agent used for the treatment of infectious diseases such as streptomycin, didanosine or zalcitabine, or any other chemically toxic agent. Infectious disease conditions such as post-polio syndrome or AIDS-associated neuropathy are specifically contemplated.

Other peripheral neuropathies include the following: HIV associated neuropathy; B-12-deficiency associated neuropathy; cranial nerve palsies; drug-induced neuropathy; industrial neuropathy; lymphomatous neuropathy; myelomatous neuropathy; multi-focal motor neuropathy; chronic idiopathic sensory neuropathy; carcinomatous neuropathy; acute pan autonomic neuropathy; alcoholic neuropathy; compressive neuropathy; vasculitic/ischaemic neuropathy; mono- and poly-neuropathies.

In further embodiments, the neuropathy is due to low back pain, Guillain-Barre Syndrome, sciatica, or other chronic pain.

Further embodiments include methods for treating a subject suffering from peripheral neuropathy, the methods comprising topically administering an effective amount of the composition comprising ketamine formulated in a pharmaceutically acceptable topical carrier.

Other embodiments include methods for treating a subject suffering from neuropathic pains, the method comprising topically administering an effective amount of a composition comprising a NMDA antagonist formulated in a pharmaceutically acceptable carrier for topical treatment.

The compositions described herein can be administered in therapeutically effective amounts. A therapeutically effective amount means the amount required to at least partly to attain the desired effect, e.g., to effectively alleviate or prevent the symptoms of the peripheral neuropathy or pain, to mitigate the side effects of certain compounds such as neurotoxicity or psychosis or drowsiness, to effectuate or potentiate the activity of the invention composition, or combinations thereof.

Such amounts will depend, of course, on the particular condition being treated, the severity of the condition, and individual patient parameters. These include age, sex, ethnicity, physical condition, size, weight, environment, sensitivity to any of the antineuropathic agents, and other concurrent treatment. The amounts prescribed will be at the discretion of the attending physician and other regulatory agents. These factors are well known to those of ordinary skill in the art, and can be addressed with no more than routine experimentation. It is generally preferred that a minimum effective dose be determined according to sound medical judgment. It will be understood by those of ordinary skill in the art, however, that a higher dose may be administered for medical, psychological or other reasons. For example, reduced dosage may be indicated in patients who are debilitated or acutely ill, in patients who are very young or very old, and in patients with liver disease, arteriosclerosis, or arterial disease.

The compositions described herein may be applied to the affected area of the skin of the patient. The frequency of application will depend on individual patient circumstances. For example, the compositions may be applied daily, twice daily, or even more frequently, as described further herein.

Methods and pharmaceutical carriers for preparation of pharmaceutical compositions, including compositions for topical administration are well known in the art, as set out in textbooks such as Remington's Pharmaceutical Sciences, 17th Edition, Mack Publishing Company, Easton, Pa., USA (updated in Gennaro, A. R., Remington: The Science and Practice of Pharmacy, 21st edition, Lippincott, Williams & Wilkins (2006)) which is incorporated by reference in its entirety.

### DETAILED DESCRIPTION

In one aspect, the pharmaceutical compositions described herein can be used for the treatment of peripheral neuropathy. These compositions can include (comprising, consisting or consisting essentially of) therapeutically effective amounts of a NMDA (receptor) antagonist in a topical vehicle that limits penetration of the NMDA antagonist generally to the skin, particularly to the epidermis, dermis and the dermatomes. Preferably, the composition comprises ketamine in amounts sufficient to treat neuropathy when the composition is administered topically in a physiologically acceptable vehicle.

As used herein, the treatment of neuropathy refers to an anti-neuropathic response or a pain-reducing response elicited through the synergistic effect of the compositions described herein, in which the combined effect of multiple agents effectively mitigates, relieves, alleviates, reduces or removes the symptoms of peripheral neuropathy, provides a beneficial effect to the subject; and/or effectively mitigates or reduces the side effects associated with the individual agents. The compositions described herein may provide one or more of the following beneficial effects to a patient when topically applied in effective amounts: relief of pain, burning, tingling, electrical sensations and/or hyperalgesia. Also increased microcirculation, nitric oxide stabilization, and facilitated healing of skin ulcers and lesions. Additionally, protein kinase C inhibition, decreased oxidative stress, anti-inflammation, protection against radiation damage (particularly ultraviolet radiation), blockage of the formation of leukotrienes, stabilization of cell membranes, and/or promotion of the synthesis of nerve growth factor.

As used herein the meaning of “NMDA-receptor antagonist” or “NMDA antagonist” encompasses compounds that block or inhibit the action of the N-methyl d-aspartate (NMDA) receptor. The receptor can be deactivated by inhibitors that can cause the NMDAR (NMDA receptor) to close by binding to allosteric sites, e.g., 1) Competitive antagonists, which bind to and block the binding site of the neurotransmitter glutamate; 2) glycine antagonists, which bind to and block the glycine site; 3) noncompetitive antagonists, which inhibit NMDARs by binding to allosteric sites; and 4) uncompetitive antagonists, which block the ion channel by binding to a site within it; or that block the NMDA receptor by another mechanism.

Examples of NMDA-receptor antagonists include, but are not limited to:

Amantadine—“Effects of N-Methyl-D-Aspartate (NMDA)-Receptor Antagonism on Hyperalgesia, Opioid Use, and Pain After Radical Prostatectomy”, University Health Network, Toronto, September 2005

Dextromethorphan—Wong B Y, Coulter D A, Choi D W, Prince D A (1988). “Dextrorphan and dextromethorphan, common antitussives, are antiepileptic and antagonize N-methyl-D-aspartate in brain slices”. Neurosci. Lett. 85 (2): 261-6.

Dextrorphan—Wong B Y, Coulter D A, Choi D W, Prince D A (1988). “Dextrorphan and dextromethorphan, common antitussives, are antiepileptic and antagonize N-methyl-D-aspartate in brain slices”. Neurosci. Lett. 85 (2): 261-6.

Ibogaine—Popik P, Layer R T, Skolnick P (1994): “The putative anti-addictive drug ibogaine is a competitive inhibitor of [3H]MK-801 binding to the NMDA receptor complex.” Psychopharmacology (Berl), 114(4), 672-4.

Ketamine—Harrison N, Simmonds M (1985). “Quantitative studies on some antagonists of N-methyl D-aspartate in slices of rat cerebral cortex”. Br J Pharmacol 84 (2): 381-91.

Nitrous oxide—Grasshoff C, Drexler B, Rudolph U, Antkowiak B (2006). “Anaesthetic drugs: linking molecular actions to clinical effects”. Curr. Pharm. Des. 12 (28): 3665-79; Kolesnikov et al. (1994) Life Sci. 55:1393. Administering inhibitors of nitric oxide synthase in morphine-tolerant animals reverses tolerance, despite continued opioid administration. Kolesnikov et al. (1993) Proc. Natl. Acad. Sci. USA 90:5162.

Phencyclidine (PCP)—

Riluzole—Hugon J (1996). “ALS therapy: targets for the future”. Neurology 47 (6 Suppl 4): S251-4.

Tiletamine—Ko J C, Smith T A, Kuo W C, Nicklin C F (1998). “Comparison of anesthetic and cardiorespiratory effects of diazepam-butorphanol-ketamine, acepromazine-butorphanol-ketamine, and xylazine-butorphanol-ketamine in ferrets”. Journal of the American Animal Hospital Association 34 (5): 407-16.

Memantine (Axura, Akatinol, Namenda, Ebixa, 1-amino-3,5-dimethylada-mantane)-Chawla, P S; Kochar M S (2006). “What's new in clinical pharmacology and therapeutics”. WMJ 105 (3): 24-29.

Dizocilpine (MK-801)—Fix A S, Horn J W, Wightman K A, et al (1993). “Neuronal vacuolization and necrosis induced by the noncompetitive N-methyl-D-aspartate (NMDA) antagonist MK(+)801 (dizocilpine maleate): a light and electron microscopic evaluation of the rat retrosplenial cortex”. Exp. Neurol. 123 (2): 204-15.

Aptiganel (Cerestat, CNS-1102)—binds the Mg2+ binding site within the channel of the NMDA receptor.

Remacimide—Muir, K W (2005). “Glutamate-based therapeutic approaches: clinical trials with NMDA antagonists”. Current Opinion in Pharmacology 6 (1): 53-60.

7-chlorokynurenate—Hartley D M, Monyer H, Colamarino S A, Choi D W (1990). “7-Chlorokynurenate Blocks NMDA Receptor-Mediated Neurotoxicity in Murine Cortical Culture”. Eur J Neurosci 2 (4): 291-295.

DCKA (5,7-dichlorokynurenic acid)—Frankiewicz T, Pilc A, Parsons C (2000). “Differential effects of NMDA-receptor antagonists on long-term potentiation and hypoxic/hypoglycaemic excitotoxicity in hippocampal slices”. Neuropharmacology 39 (4): 631-42.

Kynurenic acid—Khan M J, Seidman M D, Quirk W S, Shivapuja B G (2000). “Effects of kynurenic acid as a glutamate receptor antagonist in the guinea pig”. European archives of oto-rhino-laryngology: official journal of the European Federation of Oto-Rhino-Laryngological Societies (EUFOS): affiliated with the German Society for Oto-Rhino-Laryngology—Head and Neck Surgery 257 (4): 177-81.

1-Aminocyclopropanecarboxylic acid (ACPC)

AP7 (2-amino-7-phosphonoheptanoic acid)—van den Bos R, Charria Ortiz G, Cools A (1992). “Injections of the NMDA-antagonist D-2-amino-7-phosphonoheptanoic acid (AP-7) into the nucleus accumbens of rats enhance switching between cue-directed behaviours in a swimming test procedure”. Behav Brain Res 48 (2): 165-70.

APV (R-2-amino-5-phosphonopentanoate)—Abizaid A, Liu Z, Andrews Z, Shanabrough M, Borok E, Elsworth J, Roth R, Sleeman M, Picciotto M, Tschop M, Gao X, Horvath T (2006). “Ghrelin modulates the activity and synaptic input organization of midbrain dopamine neurons while promoting appetite”. J Clin Invest 116 (12): 3229-39.

CPPene (3-[(R)-2-carboxypiperazin-4-yl]-prop-2-enyl-1-phosphonic acid)—Eblen F, Loschmann P, Wüllner U, Turski L, Klockgether T (1996). “Effects of 7-nitroindazole, NG-nitro-L-arginine, and D-CPPene on harmaline-induced postural tremor, N-methyl-D-aspartate-induced seizures, and lisuride-induced rotations in rats with nigral 6-hydroxydopamine lesions”. Eur J Pharmacol 299 (1-3): 9-16.

Lacosamide

Other NMDA-receptor antagonists include, but are not limited to, eliprodil; iamotrigine; flupirtine; celfotel; levemopamil; pyroloquinoline quinone; cis-4-(phosphonomethyl)-2-piperidine carboxylic acid; 1-(4-hydroxy-phenyl)-2-(4-phenylsulfanyl-piperidin-1-yl)-pro pan-1-one; 2-[4-(4-fluoro-benzoyl)-piperidin-1-yl]-1-naphthalen-2-yl-et hanone (E 2001); 3-(1,1-dimethyl-heptyl)-9-hydroxymethyl-6,6-dimethyl-6a,7,8,10a-tetrahydro-6H-benzo[c]chromen-1-ol (HU-211); 1-{4-[1-(4-chloro-phenyl)-1-methyl-ethyl]-2-methoxy-phenyl}-1H-[1,2,4]triazole-3carboxylic acid amide (CGP 31358); acetic acid 10-hydroxy-7,9,7′,9′-tetramethoxy-3,3′-dimethyl-3,4,3,4′-tetrahydro-1H,1′H-[5,5′]bi[benzo[g]isochromenyl]-4-yl ester (ES 242-1); 14-hydroxy-11-isopropyl-10-methyl-5-octyl-10,13-diaza-tricyclo[6.6.1.04,15]pentadeca-1,4,6,8 (15)-tetraen-12-one; and 4,5-dioxo-4,5-dihydro-1H-benzo[g]indole-2,7,9-tricarboxylic acid (PQQ), 3-((−)-2carboxypiperazin-4-ylpropyl-1-phosphate (CPP); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4phosphonomethyl-phenyl)-acetic acid (PD 129635); 2-amino-3-(5-chloro-1phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6phosphonomethyl-decahydro-isoquinoline-3-carboxylic acid (LY 274614); 4-(1H-tetrazol-5ylmethyl)-piperidine-2-carboxylic acid (LY 233053 and 235723); 6-(1H-Tetrazol-5ylmethyl)-decahydro-isoquinoline-3-carboxylic acid (LY 233536); phencyclidine; thienylcyclohexylpiperidine (TCP); N-allylnormetazocine (SKF 10,047); cyclazocine; (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558; and quinoxalinediones, such as 6-cyano-7-nitroquinoxaline-2,3-dione (CNQX); 6,7-dinitro-quinoxaline-2,3-dione (DNQX); spermine; spermidine; putrescine; arcaine; PEAQX; PPDA; hodgkinsine; dexoxadrol; endopsychosin; etoxadrol; eticyclidine,; rhynchophylline; rolicyclidine; tenocyclidine; xenon; 7-chlorokynurenate; CGP-39653; DCKA; kynureneic acid; L-689,560; CP-101,606; ifenprodil; Ro25-6981; and the like. References that disclose other NMDA-receptor antagonists as well as assays for identifying NMDA-receptor antagonists include Jia-He Li, et al., 38 J. Med. Chem. 1955 (1995); Bigge 45 Biochem. Pharmacol. 1547 (1993); Steinberg et al, 133 Neurosci. Lett. 225 (1991); Meldrum et al., 11 Trends Pharmacol. Sci., 379 (1990); Willetts et al., 11 Trends Pharmacol. Sci. 423 (1990); Faden et al., 13 Trends Pharmacol. Sci. 29 (1992); Rogawski 14 Trends Pharmacol. Sci. 325 (1993); Albers et al, 15 Clinical Neuropharm. 509 (1992); Wolfe et al., 13 Am. J Emerg. Med., 174 (1995); and Bigge, 45 Biochem. Pharmacol. 1547 (1993); U.S. Pat. No. 6,251,948 (issued Jun. 26, 2001); U.S. Pat. No. 5,985,586 (issued Nov. 16, 1999), and U.S. Pat. No. 6,025,369 (issued Feb. 15, 2000); Jacobson et al., 110 J. Pharmacol. Exp. Ther. 243 (1987); and Thurkauf et al., 31 J. Med. Chem. 2257 (1988), all of which citations are hereby expressly incorporated herein by reference.

Testing NMDA-receptor antagonist for local-analgesic/anesthetic and peripheral antinociceptive properties according to standard pain models can identify NMDA-receptor antagonist suitable for use in the invention, including using the compounds disclosed above or derivatives thereof. See e.g., J. Sawynok et al., 82 Pain 149 (1999); J. Sawynok et al., 80 Pain 45 (1999). Preferred NMDA antagonists contemplated by the present invention are compounds with analgesic properties readily formulated for topical applications and use. More preferred are compounds that are clinically or pharmaceutically available.

Preferably, the NMDA antagonist is a non-competitive channel blocker such as amantadine, dextromethorphan, ibogaine, ketamine, norketamine, memantin, riluzole, tiletamine, dextrorphan, and phencyclidine. More preferred are compounds with NMDA receptor antagonist activities that are existing pharmaceuticals or nutraceuticals (e.g., have undergone one or more regulatory trials in humans or animals (e.g., FDA based Phase I, Phase II and/or Phase III trials). More preferably, the NMDA-receptor antagonist is a non-competitive NMDA-receptor antagonist, more preferably, ketamine, even more preferably, ketamine hydrochloride.

The amount of NMDA-receptor antagonist in compositions of the invention will vary according to the type and identity of the NMDA-receptor antagonist, the pain indication treated, and such other factors as described herein. Dosages and concentrations for a particular NMDA-receptor antagonist in the invention composition can be optimized according to routine experiments using well-known pain models, for example, those described in J. Sawynok et al., 82 Pain 149 (1999) and J. Sawynok et al., 80 Pain 45 (1999).

Ketamine is an N-methyl-D-aspartate (NMDA) calcium channel antagonist that can be admixed in the compositions described herein in concentrations ranging from 10-50%, preferably 10 to 30%, and most preferably from 15% to 20% safely to 25%. Topical ketamine is effective for treating painful neuropathy when other traditional medicines have failed. See Crowley K L, Flores J A, Hughes C N et al. “Clinical application of ketamine ointment in the treatment of sympathetically maintained pain”, International Journal of Pharmaceutical Compounding 1998; 2:122-127.

Certain NMDA antagonists have significant side effects, including psychotomimetic conditions. In adult humans, certain NMDA produce psychosis in humans and neurotoxicity in animals (see, e.g., Hargreaves R, Hill R, Iversen L. “Neuroprotective NMDA antagonists: the controversy over their potential for adverse effects on cortical neuronal morphology”. Acta Neurochir Suppl (Wien) 60: 15-9; Olney J, Labruyere J, Price M (1989). “Pathological changes induced in cerebrocortical neurons by phencyclidine and related drugs”. Science 244 (4910): 1360-2; Anderson C. “The Bad News Isn't In: A Look at Evidence for Specific Mechanisms of Dissociative-Induced Brain Damage and Cognitive Impairment”. Erowid.org, June 2003: http://www.erowid.org/chemicals/dxm/dxm_health2.shtml); Graeme E. Correll, Jahangir Maleki, Edward J. Gracety, Jesse J. Muir, Ronald E. Harbut (2004). “Subanesthetic Ketamine Infusion Therapy: A Retrospective Analysis of a Novel Therapeutic Approach to Complex Regional Pain Syndrome.” Pain Medicine 5(3):263-275.

The topical pharmaceutical compositions described herein can further comprise alternative NMDA receptor antagonists, as an alternative to ketamine or as a supplemental analgesic. These antagonists can be competitive or non-competitive drugs. The NMDA receptor antagonist can be any known in the art, including, but not limited to dextromethorphan, dextrorphan, pyroloquinoline quinone, cis-4-(phosphonomethyl)-2-piperidine carboxylic acid, MK801, memantine, and their mixtures and pharmaceutically acceptable salts thereof.

In general, the amount of NMDA-receptor antagonist in the compositions of the invention is within the range of from about 0.1 percent to about 50 percent of the total weight of the composition, more preferably, of from about 3 percent to about 30 percent of the total weight of the composition. More preferably, the range is from about 10 percent to about 30 percent of the total weight, and includes the ranges, 11, 12, 13, 14, 15, 16, 17, 18, 19, 20, 21, 22, 23, 24, 25, 26, 27, 28, 29, and 30 percent. For example, the amount of ketamine in the invention composition is within the range of from about 5% to about 40%, more preferably from about 10% to about 40, more preferably from about 15% to about 30%, even more preferably from about 20% to about 25%, or alternatively, greater than 10% or 15%. Such ranges of NMDA antagonist employed in the present invention would likely, prior to applicant's disclosure, be considered too high for clinical application, e.g., too high for one time use or, alternatively, too high for repeated use on a large population of patients.

In addition, the compositions described herein can further comprise additional ingredients that can increase the analgesic effectiveness of the combination of invention composition. Such ingredients either facilitate the effect of the present invention by minimizing absorption and/or penetration to the skin, providing for or enhancing a local pain management regimen, decreasing the systemic effect of the NMDA antagonist, enhancing NMDA receptor antagonism, or the like. For example, magnesium ions (e.g., from magnesium oxide or other magnesium preparations) antagonize ionic calcium in the nervous system, enhancing the effect of the present invention. Those of skill in the art will readily recognize additional ingredients that can be admixed in the compositions described herein.

Co-administration of a magnesium salt is also preferred, and apparently can increase the pain-relieving efficacy of this treatment in at least some cases. As used herein, the term “salt” includes any compound or complex that releases substantial quantities of free magnesium ions (Mg++) when dissolved in an aqueous solution.

Nerve impulse conduction is blocked by a decrease in nerve cell membrane permeability to sodium ions, possibly by competing with calcium-binding sites that control sodium permeability. This change in permeability results in decreased depolarization and an increased excitability threshold that ultimately prevents the nerve action potential from forming.

Ionic calcium is antagonized by magnesium ions in the nervous system. Because of this, dietary supplements of magnesium oxide and other magnesium preparations may increase or enhance the effects of calcium channel blockade.

Magnesium can effect muscle relaxation through direct action on the cell membrane. Mg++ ions close certain types of calcium channels, which conduct a positively charged calcium ion into the neuron. With an excess of magnesium, more channels will be blocked and the nerve will have less activity.

The compositions described herein can further comprise components usually admixed in such preparations (besides a NMDA antagonist). For example, the compositions may also include additional ingredients such as other carriers, moisturizers, oils, fats, waxes, surfactants, thickening agents, antioxidants, viscosity stabilizers, chelating agents, buffers, preservatives, perfumes, dyestuffs, lower alkanols, humectants, emollients, dispersants, sunscreens such as radiation blocking compounds or particularly UV-blockers, antibacterials, antifungals, disinfectants, vitamins, antibiotics, or other anti-acne agents, as well as other suitable materials that do not have a significant adverse effect on the activity of the topical composition. Additional ingredients for inclusion in the carrier are sodium acid phosphate moisturizer, witch hazel extract carrier, glycerin humectant, apricot kernel oil emollient, corn oil dispersant, and the like which are further detailed below. Those of skill in the art will readily recognize additional ingredients, which can be admixed in the compositions described herein.

In addition to the foregoing components, the compositions described herein can optionally contain other ingredients. For example, triethanolamine can be added as a crosslinking agent. A preservative, such as betahydroxytoluene can also be added. Other irritation reducing agents can be added too. In this regard, irritation reducing agents can include, but are not limited to, glycerol. In some instances, semi-solid testosterone formulations have been prepared with propylene glycol and/or butylene glycol as the glycol component, ethyl alcohol and/or isopropyl alcohol as the alcohol component. Preservatives, a cross-linking agent, and additional irritation reducing agents can be included in formulations prepared in accordance with the methods described.

The compositions and methods of the invention are effective to induce local anaelgesia and to treat neuropathic pain. As used herein the term “neuropathic pain” refers to neuropathic-pain syndromes, that is, pain due to lesions or dysfunction in the nervous system. As used herein, the term “local” refers to the limited area near the site of administration, generally the nerves at or near skin including the epidermis, the dermis, the dermatomes and the like. Local analgesics of the present invention reversibly block nerve conduction near their site of administration, thereby producing temporary loss of sensation and/or relief of pain or neuropathy in a limited area (the nerves of the dermis or dermatome (area of the skin associated with dorsal roots from the spine)), with no or limited systemic penetration beyond the skin.

The compositions and methods of the invention can be used to treat or prevent pain related to or induced by the following diseases, trauma, or conditions: general neuropathic conditions, such as peripheral neuropathy, phantom limb pain, reflex-sympathetic dystrophy, causalgia, syringomyelia, and painful scar; specific neuralgias at any location of the body; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; connective tissue disorders, such as systemic lupus erythematosis, systemic sclerosis, polymyositis, and dermatomyositis; and inflammatory conditions, such as acute inflammation (e.g. trauma, surgery and infection) or chronic inflammation (e.g., arthritis and gout).

Topical application of the composition may be useful for relieving pain, inflammation and irritation associated with skin diseases and disorders, such as psoriasis, pruritus, and lesions. Painful lesions develop, for example, from viral infections, skin cancers and genetic disorders. Topical application of the composition provides relief from pain associated with wounds, insect and animal bites, abrasions and burns, including those resulting from over-exposure to the sun, chemicals, radiation or chemotherapeutic agents. Acute post-operative or surgical pain can be reduced or even prevented, as can pain associated with chronic disorders, such as arthritis.

In preferred embodiments the methods described herein can provide a treatment of applying the compositions described herein to an affected area of a subject with diabetic polyneuropathy. In other aspects, the methods described herein can include treating peripheral neuropathy, comprising the step of topical administration of a pharmaceutical composition of ketamine in a topical vehicle to the affected area of a subject in need of such treatment.

Thus, the methods and compositions described herein can be effective for neuropathies, particularly peripheral neuropathies, associated with diseases such as: uremia; childhood cholestatic liver disease; chronic respiratory insufficiency; alcoholic polyneuropathy; multiple organ failure; sepsis; hypoalbuminemia; eosinophilia-myalgia syndrome; hepatitis; porphyria; hypoglycemia; vitamin or nutritional deficiency (e.g., B-12 deficiency); chronic liver disease; primary biliary cirrhosis; hyperlipidemia; leprosy; Lyme disease; herpes zoster; Guillain-Barre syndrome; chronic inflammatory demyelinating polyradiculoneuropathy; sensory perineuritis; HIV or acquired immunodeficiency syndrome (AIDS)-associated neuropathy; Sjogren's syndrome; primary vasculitis (such as polyarteritis nodosa); allergic granulomatous angiitis; hypersensitivity angiitis; Wegener's granulomatosis; Raynaud's Phenomenon, including CREST syndrome, autoimmune diseases such as erythromatosis (systemic lupus erythematosis); rheumatoid arthritis or other rheumatoid diseases; mixed connective tissue disease; scleroderma; sarcoidosis; vasculitis; systemic vasculitides; acute tunnel syndrome; pandysautonomia; primary, secondary, localized or familial systemic amyloidosis; hypothyroidism; chronic obstructive pulmonary disease; acromegaly; malabsorption (sprue, celiac disease); carcinomas (sensory, sensorimotor, late and demyelinating); lymphoma (including Hodgkin's), polycythemia vera; multiple myeloma (lytic type, osteosclerotic, or solitary plasmacytoma); benign monoclonal gammopathy; macroglobulinemia; cryoglobulinemia; tropical myeloneuropathies; herpes simplex infection; cytomegalovirus infection; cranial nerve palsies; drug-induced neuropathy; industrial neuropathy; lymphomatous neuropathy; myelomatous neuropathy; multi-focal motor neuropathy; immune-mediated disorders, chronic idiopathic sensory neuropathy; carcinomatous neuropathy; acute pain autonomic neuropathy; alcoholic neuropathy; compressive neuropathy; vasculitic/ischaemic neuropathy; mono- and polyneuropathies; and diabetes.

Genetically acquired neuropathies suitable for treatment by the methods and compositions described herein include, without limitation: peroneal muscular atrophy (Charcot-Marie-Tooth Disease) hereditary amyloid neuropathies, hereditary sensory neuropathy (type I and type II), porphyric neuropathy, hereditary liability to pressure palsy, Fabry's Disease, adrenomyeloneuropathy, Riley-Day Syndrome, Dejerine-Sottas neuropathy (hereditary motor-sensory neuropathy-III), Refsum's disease, ataxia-telangiectasia, hereditary tyrosinemia, anaphalipoproteinemia, abetalipoproteinemia, giant axonal neuropathy, metachromatic leukodystrophy, globoid cell leukodystrophy, and Friedrich's ataxia.

In alternative embodiments compositions described herein are directed to treatment of neuropathic pain, especially pain caused by nerve injury or sympathetically mediated pain. Sympathetically mediated pain (SMP) is a type of pain in which over activity of the sympathetic nervous system plays a crucial role. It includes the syndromes of reflex sympathetic dystrophy (RSD), causalgia, neuropathic pain secondary to nerve injury, and pain from neuromas. It encompasses all neurogenic pain that is not central and is related to a nerve injury regardless of the cause. Neuropathic pain syndromes include, without limitation (other than the neuropathies described herein), allodynia, various neuralgias such as post herpetic neuralgia and trigeminal neuralgia, phantom limb pain, hyperpathia, hyperesthesia, hyperalgesia, dyesthesia, paresthesia, anesthesia delorosa, deafferatation pain, and complex regional pain syndromes (CRPS), such as reflex sympathetic dystrophy (RSD) and causalgia. Specific examples include low back pain, sciatica, Guillain-Barre Syndrome, post-surgical traumatic neuropathy, and diabetic peripheral polyneuropathy.

Formulations

As described herein, the present invention comprises a therapeutically effective amount of a NMDA (receptor) antagonist in a topical vehicle that limits penetration of the NMDA antagonist generally to the skin, particularly to the epidermis, dermis and the dermatomes, or alternatively minimizes systemic penetration. Preferably, the composition comprises ketamine in amounts sufficient to treat neuropathy when the composition is administered topically in a physiologically acceptable vehicle or carrier. The formulations in which the compositions described herein are incorporated can assume any of a variety of dosage forms, including solutions, suspensions, ointments, and solid inserts. Examples are creams, lotions, gels, ointments, suppositories, sprays, foams, liniments, aerosols, buccal and sublingual tablets, various passive and active topical devices for absorption through the skin and mucous membranes, including topical applications, and the like.

Preferably, the topical delivery is designed to maximize drug delivery through the stratum corneum and into the epidermis or dermis or dermatome, and to minimize absorption into the circulatory system. More preferable are agents that may be used in topical formulations to prevent the passage of active ingredients or excipients into the lower skin layers. These so-called skin retardants have been readily developed for many over-the-counter (OTC) skin formulations, such as sunscreens and pesticides, where the site of action is restricted to the skin surface or upper skin layers. Research in the area of permeation enhancement or retardation is yielding valuable insights into the structure-activity relationships of enhancers as well as retardants (See, e.g., Asbill C S and Michniak B B. 2000. Pharm Sci & Tech Today, 3(1):36-41; Kaushik D. et al., 2008. Exp Opin Drug Del. 5(5):517-529; Trommer H and Neubert R H H. 2006. Skin Pharmacol Physiol 19:106-121; Neubert R et al., 1996. Pharmazeutische Zeitung 141(17):1483-1493; Benson H A E, 2005 Curr Drug Del 2(1):23-33) including such compounds as ketorolac stearate, Aminocaprolactam Analogues, Dicarboxylic acid ester, sodium citrate, and the like.

Typical pharmaceutically acceptable carriers are, for example, water, mixtures of water and water-miscible solvents such as lower alkanols or vegetable oils, and water-soluble ophthalmologically acceptable non-toxic polymers, for example, cellulose derivatives such as methylcellulose. A typical cream or ointment-type carrier for topical application that can be used according to the methods and compositions described herein include a mixture of water, glycerin, propylene glycol, and methylparaben. Topical carriers may also include other conventional emulsifiers and emollients including alginates, glyceryl stearate, PEG-100 stearate, cetyl alcohol, propylparaben, butylparaben, sorbitols, polyethoxylated anhydrosorbitol monostearate (TWEEN), white petrolatum (VASELINE), triethanolamine, Emu oil, aloe vera extract, lanolin, cocoa butter, and the like. Suitable topical carriers are well known to the skilled artisan. Standard texts, such as Gennaro, A. R., Remington: The Science and Practice of Pharmacy, 21st edition, Lippincott, Williams & Wilkins (2006), J. G., Goodman & Gilman's The Pharmacological Basis of Therapeutics, 10th edition, McGraw-Hill Professional; Shah & Maibach, Topical Drug Bioavailability, Bioequivalence, and Penetration, 1st edition, Plenum Pub Corp.; Hillery et al., Drug Delivery and Targeting: For Pharmacists and Pharmaceutical Scientists, Harwood Academic Pub.; Ansel et al., Pharmaceutical Dosage Forms and Drug Delivery Systems, 7th edition, Lippincott Williams & Wilkins (each incorporated herein by reference), can be consulted to prepare suitable compositions for topical administration, without undue experimentation. Suitable dosages can also be determined based upon the text and documents cited herein.

Examples of solvents or solubilizers which may comprise the pharmaceutically acceptable vehicle of this invention include one or more of materials such as glycerin, propylene glycol, isopropanol, ethanol, a variety of polyethylene glycols, block copolymers of ethylene glycol and propylene glycol, acetylated monoglycerides, lanolin, mineral oil, water, aqueous buffers and the like.

The lipophilic component in the compositions of the invention can be any water insoluble (hydrophobic) organic material or mixture of materials that can form a stable emulsion comprising an NMDA-receptor antagonist suitable for intradermal administration. Preferably, the lipophilic component comprises about 15% to about 40% by weight of the total composition weight, more preferably, about 20% by weight.

Suitable lipophilic components are well known in the art and include, but are not limited to, vegetable, nut, and seed oils, such as almond oil, castor oil, coconut oil, corn oil, cotton seed oil, jojoba oil, linseed oil, grape seed oil, rape seed oil, mustard oil, olive oil, palm and palm kernel oil, peanut oil, safflower oil, sesame oil, soybean oil, sunflower-seed oil, crambe oil, wheat germ oil, and cocoa butter; animal oils and fats, such as lanolin, tallow, lard, beef fat, butterfat, mink oil, and fish oils; hydrocarbon and petroleum oils, such as petrolatum, mineral oil, and liquid paraffin; and higher fatty acids such as lauric acid, myristic acid, palmitic acid, stearic acid, behenic acid, oleic acid, 12-hydroxystearic acid, undecylenic acid, tall acid, lanolin fatty acid, isostearic acid, linoleic acid, and linolenic acid, and derivatives thereof. Preferably, the lipophilic component is a petroleum oil, such as petrolatum, mineral oil, or liquid paraffin, more preferably, petrolatum.

Preferably, the lipophilic component further comprises a “stiffening agent” (i.e., a hydrophobic material that is a solid at room temperature but melts within the temperature range of about 40° C. to 80° C.) to provide a creamy feel to the compositions of the invention. The preferred amount of stiffening agent is about 1% to about 10% by weight of the total composition weight. Examples of suitable stiffening agents include, but are not limited to, cetyl alcohol, cetyl esters wax, microcrystalline wax, paraffin, stearyl alcohol, lauryl alcohol, miracle alcohol, cetostearyl alcohol, white wax, yellow wax, bee wax, candelilla wax, cotton wax, carnauba wax, bayberry wax, rice-bran wax. Cetyl alcohol is the preferred stiffening agent.

Preferably, the lipophilic component further comprises a hydrophobic material that facilitates absorption of the NMDA-receptor antagonist into the skin, referred to herein as a “lipophilic intradermal-penetration enhancer”. The preferred amount of lipophilic-intradermnal-penetration enhancer is about 1% to about 99% by weight, more preferably about 1% to about 15%, of the total composition weight. Suitable lipophilic intradermal penetration enhancers include isopropyl myristate, glycerol monolaurate, glycerol monooleate, glycerol monolinoleate, isopropyl isostearate, isopropyl linoleate, isopropyl myristate/fatty acid monoglyceride combination, isopropyl myristate/ethanol/L-lactic acid combination, isopropyl palmitate, methyl acetate, methyl caprate, and methyl laurate.

The compositions of the invention comprise a surfactant to stabilize the emulsion. Surfactants can be cationic, nonionic, anionic, or amphoteric. For an extensive discussion on surfactants and emulsions, see Gillian M. Eccleston, Emulsions in 5 Encyclopedia of Pharmaceutical Technology 137-184 (James C. Swarbrick & James C. Boylan eds. 1988). For use in the invention, the surfactant can be any intradermally-acceptable hydrophilic or hydrophobic material or mixture of materials capable of stabilizing an oil-in-water type emulsion. One of skill in the art will readily choose a suitable surfactant or surfactant mixture based on the hydrophilic-lipophilic balance (HLB) values of the surfactant and the lipophilic component. The preferred amount of surfactant is about 2% to about 15% by weight of the total weight of the composition, more preferably, about 10%.

Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinate, disodium lauryl sulfosuccinate, diammonium lauryl sulfosuccinate, tetrasodium N-(1,2-dicarboxyethyl)-N-octadecylsulfosuccinate, diamyl ester of sodium sulfosuccinic acid, dihexyl ester of sodium sulfosuccinic acid, dioctyl esters of sodium sulfosuccinic acid, docusate sodium, and combinations thereof.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof. Also included are Cremophor RH 40TM (polyoxyl 40 hydrogenated castor oil), Cremophor ELTM (polyoxyl 35 castor oil), Cremophor ELPTM (polyoxyl 35 castor oil), and Solutol HS 15TM (macrogol 15 hydroxystearate), PEG-40 palm kernel oil, PEG-50 hydrogenated castor oil, PEG-60 castor oil, monostearate (and derivatives thereof), glyceryl laurate, glyceryl stearate, glyceryl oleate, glyceryl monooleate, glyceryl monolaurate, sorbitan monooleate, sorbitan monolaurate, sorbitan monopalmitate, sorbitan stearate, nonyl phenols, octyl phenols, caprylocaproyl polyoxyglycerides, lauroyl polyoxyglycerides, stearoyl polyoxylglycerdes and d-α-tocopheryl polyethylene glycol succinate, or combinations thereof. More preferably, the non-ionic surfactant is Cremophor RH 40TM (polyoxyl 40 hydrogenated castor oil), Cremophor ELTM (polyoxyl 35 castor oil), Cremophor ELPTM (polyoxyl 35 castor oil), Solutol HS 15TM (macrogol 15 hydroxystearate) or TPGS™ (d-α-tocopheryl polyethylene glycol succinate). Most preferably, the non-ionic surfactant is Cremophor RH 40TM (polyoxyl 40 hydrogenated castor oil), Cremophor ELTM (polyoxyl 35 castor oil) or Cremophor ELPTM (polyoxyl 35 castor oil), and the like

Examples of amphoteric surfactants include, but are not limited to, sodium N-dodecyl-γ-alanine, sodium N-lauryl-γ-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and combinations thereof.

Examples of cationic surfactants include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, laurtrimonium chloride, methyl-1-oleyl amide ethyl-2-oleyl imidazolinium methyl sulfate, picolin benzyl ammonium chloride, polyquatemium, stearalkonium chloride, sterayl dimethylbenzyl ammonium chloride, stearyl trimethyl ammonium chloride, trimethylglycine, and combinations thereof.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays are conveniently delivered from pressurized packs, for example, via a specially shaped closure. Oil-In-Water emulsions can also be utilized in the compositions, patches, bandages and articles. These systems are semisolid emulsions, micro-emulsions, or foam emulsion systems. Usually such a system has a “creamy white” appearance. The oleaginous phase may contain, but is not limited to, long-chain alcohols (cetyl, stearyl), long-chain esters (myristates, palmitates, stearates), long-chain acids (palmitic, stearic), vegetable and animal oils and assorted waxes. These can be made with anionic, cationic, nonionic or amphoteric surfactants, or with combinations especially of the nonionic surfactants.

Preferably, Lipoderm® (Professional Compounding Centers of America, Houston, Tex.) is admixed in the compositions described herein. Alternative ointment bases are known to persons skilled in the art such as Transcutol-P (ethoxydiglycol, commercially available, for example, from Gattefosse, Westwood, N.J.). Sufficient Lipoderm® base is admixed to act as a carrier for the active ingredients of the composition. Typically the Lipoderm® base will make up more than about 70% of the total composition and more preferably about 74% of the composition is the Lipoderm® base. The Lipoderm® base functions as a carrier and enhances penetration through the skin. It is also hypoallergenic and is aesthetically pleasing.

A typical invention gel base, provided herein for exemplary purposes only, can contain lecithin, isopropyl palmitate, poloxamer 407, and water. Topical carriers with different viscosities and hand-feel are known to the art. The above active ingredients can be dispersed within the pharmaceutically acceptable carrier in therapeutically effective amounts to treat neuropathies, and the other maladies described above. Preferably, the topical pharmaceuticals described herein contain (per gram total weight) from about 15 grams to 30 grams per 100 grams (more preferably 20 grams, 25 grams or 30 grams) weight of a NMDA antagonist. Other agents can be added accordingly.

Topical dosage unit forms can be prepared utilizing a variety of techniques that have been described in the art. For example, in U.S. Pat. Nos. 4,861,800; 4,868,218; 5,128,145; 5,190,763; and 5,242,950; and in the foreign patent documents EP-A 404807; EP-A 509761; and EP-A 593807 (each of which is incorporated by reference in its entirety). A monolithic patch structure can be utilized in which selegiline is directly incorporated into the adhesive and this mixture is cast on to a backing sheet. Alternatively, selegiline as an acid addition salt can be incorporated into a multi layer patch which effects a conversion of the salt to selegiline-free base, as described for example in EP-A 593807. One can also employ a device using a lyotropic liquid crystalline composition in which, for example, 5-15% of selegiline is combined with a mixture of liquid and sold polyethylene glycols, a polymer, and a non-ionic surfactant, optionally with the addition of propylene glycol and an emulsifying agent. For further details on the preparation of such topical formulations, reference can be made to EP-A 509761.

“Drug delivery system,” “drug/enhancer composition,” or any similar terminology relates to a formulated composition containing the drug to be topically delivered in combination with a penetration enhancer. Other pharmaceutically acceptable materials or additives can also be contained in the drug/enhancer composition, such as a diluent, skin-irritation reducing agent, carrier or vehicle, excipient, plasticizer, emollient, or other additive and mixtures thereof provided that such additives do not materially affect the basic and novel characteristics of the matrix patch.

The terms “matrix,” “matrix system,” or “matrix patch” relate to an active permeant or drug dissolved or suspended in a biocompatible polymeric phase, preferably a pressure sensitive adhesive, that can also contain other ingredients or in which the enhancer is also dissolved or suspended. This definition is meant to include embodiments wherein such polymeric phase is laminated to a pressure sensitive adhesive or used with an overlay adhesive. A matrix system usually and preferably comprises an adhesive layer having an impermeable film backing laminated onto the distal surface thereof and, before topical application, a release liner on the proximal surface of the adhesive. The film backing protects the polymeric phase of the matrix patch and prevents release of the drug and/or enhancer to the environment. The release liner functions similarly to the impermeable backing, but is removed from the matrix patch prior to application of the patch to an application situs. Matrix patches are known in the art of topical drug delivery to routinely contain such backing and release liner components, and matrix patches according to the compositions described herein should be considered to comprise such backing and release liner or their functional equivalents. U.S. Pat. No. 5,122,383 (incorporated herein by reference) describes such backing and release liner. A matrix system therefore relates to a unit dosage form of a drug composition in a polymeric carrier, also containing the enhancer and other components that are formulated for maintaining the drug composition in the polymeric layer in a drug transferring relationship with the derma, i.e. the skin or mucosa. A matrix patch is distinguished from a “liquid reservoir patch,” wherein an active permeant or drug is dissolved in a gelled liquid contained in an occlusive device having an impermeable back surface and an opposite surface configured appropriately with a permeable membrane and adhesive for topical application, e.g., U.S. Pat. No. 4,983,395, incorporated herein by reference in its entirety.

A typical topical formulation comprises a conventional aqueous or non-aqueous vehicle, for example, a cream, ointment lotion or paste or in the form of a medicated plaster, patch or membrane.

The term “effective amount” of a drug or permeant relates to a nontoxic but sufficient amount of a compound to provide the desired local or systemic effect without adverse side effects. An “effective amount” of permeation enhancer as used herein relates to an amount selected so as to provide the desired increase in membrane permeability and, correspondingly, the desired depth of penetration, rate of administration, and amount of drug.

As used herein, “application situs” relates to a site suitable for topical application with or without the means of a device, patch, or dressing, e.g. the spinal column, behind the ear, on the arm, back, chest, abdomen, leg, top of foot, etc. For example, the cream can be applied to the site of pain or the pain site of spine dermatome(s), e.g., l2-s2 for any leg, knee, or foot neuropathy.

The penetration enhancing compositions of the compositions described herein may constitute a small amount of the formulation or a large amount depending on which topical vehicle is used, which systemically and/or topically active agent is used, and the type of biological effect sought. The amount will be readily apparent to those skilled in the art, since the total amount of penetration enhancers will be approximately the same as those of the prior art. For example, when the potency of the penetration enhancement composition is greatly increased, lower quantities can be used.

As used herein, topical delivery also includes numerous different systems for the topical delivery of active agents known in the art. Topical delivery systems include but are not limited to passive devices such as drug-in-adhesive topical patches and “active” topical technologies such as iontophoresis, electroporation, sonophoresis, magnetophoresis, microneedle devices and those devices that use thermal energy to make the skin more permeable.

Topical drug delivery devices are available from the 3M Drug Delivery Systems Division (St. Paul, Minn., USA), Noven Pharmaceuticals, Inc. (Miami, Fla., USA), ImaRx (Tucson, Ariz., USA), Elan Corporation (Dublin, Ireland), Novosis AG (Miesbach, Germany), Ultrasonic Technologies (St. Albans, Vt., USA), Antares Pharma (Exton, Pa., USA), Altea Therapeutics (Tucker, Ga., USA), Iomed, Inc. (Salt Lake City, Utah, USA), MacroChem Corp (Lexington, Mass., USA), Sontra Medical Corporation (Franklin, Mass., USA), Vyteris, Inc. (Fair Lawn, N.J., USA), BioChemics, Inc. (Danvers, Mass., USA), A.P Pharma (Redwood, City, Calif., USA), MIKA Pharma GmbH (Limburgerhof, Germany), NexMed, Inc. (Robbinsville, N.J., USA), Encapsulation Systems, Inc. (Springfield, Pa., USA), Acrux Ltd (Elgin, Ill., USA), Jenapharm GmbH (Berlin, Germany), Norwood Abbey (Victoria, Australia), Novavax (Columbia, Md., USA), Genetronics Biomedical Corporation (San Diego, Calif., USA), Adherex Technologies (Research Triangle Park, N.C., USA), and AlphaRx (Ontario, Canada).

Topical drug delivery using patch technology is typically accomplished by using a covering element in the form of a topical patch device that is attached to the host at the desired drug delivery site. A typical topical patch structure includes a drug-in-adhesive layer sandwiched between an impermeable backing and a release liner. At the time of use, the release liner is easily removed so that the patch can be attached to the host, adhesive side down. The impermeable backing thus traps the drug-in-adhesive layer between the backing and the attachment site of the host. Over time, the drug penetrates into the host, or is topically active, in accordance with the desired therapeutic treatment. Optionally, the drug-in-adhesive formulation may include one or more compounds known as penetration enhancers that increase the delivery of the drug to the subject. (See U.S. Pat. No. 6,627,216).

Some examples of topical patch technology include but are not limited to those described in U.S. Pat. No. 6,592,893; U.S. Pat. No. 6,267,983 to Fuji et al.; U.S. Pat. No. 6,238,693 to Luther et al.; U.S. Pat. No. 6,211,425 to Takayasu et al.; U.S. Pat. No. 6,159,497 to LaPrade et al.; U.S. Pat. No. 6,153,216 to Cordes et al.; U.S. Pat. No. 5,948,433 to Burton et al.; U.S. Pat. No. 5,508,035 to Wang et al.; U.S. Pat. No. 5,284,660 to Lee et al.; U.S. Pat. No. 4,942,037 to Bondi et al.; and U.S. Pat. No. 4,906,463 to Cleary et al.

Iontophoresis, an active topical technology, uses low voltage electrical current to drive charged drugs through the skin. Those molecules with a positive charge are driven into the skin at the anode and those with a negative charge are driven into the skin at the cathode. See U.S. Pat. No. 6,622,037 to Kasamo. Additional examples of iontophoretic delivery devices for the topical delivery of active agents include but are not limited to those described in U.S. Pat. No. 6,564,903 to Ostrow et al.; U.S. Pat. No. 5,387,189 to Gory et al; U.S. Pat. No. 5,358,483 to Sibalis; U.S. Pat. No. 5,356,632 to Gross et al; U.S. Pat. No. 5,312,325 to Sibalis; U.S. Pat. No. 5,279,544 to Gross et al; U.S. Pat. No. 5,167,479 to Sibalis; U.S. Pat. No. 5,156,591 to Gross et al, U.S. Pat. No. 5,135,479 to Siballs et al; U.S. Pat. No. 5,088,977 to Sibalis; U.S. Pat. No. 5,057,072 to Phipps; U.S. Pat. No. 5,053,001 to Reller et al; and U.S. Pat. No. 4,942,883 to Newman.

Electroporation is similar to iontophoresis in that it uses electrical fields to aid in transport of molecules across the stratum corneum. However, rather than driving the molecules through the skin, electroporation uses high-voltage electric field pulses to create transient pores which permeabilize the stratum corneum (SC) (Prausnitz et al., Proc. Natl. Acad. Sci. 90:10504-10508 (1993); Murthy et al. J. Control. Release 98:307-315 (2004); U.S. Pat. No. 5,947,921)). Examples of electroporation technology for topical delivery include but are not limited to U.S. Pat. No. 6,692,456 to Eppstein et al.; U.S. Pat. No. 6,564,093 to Ostrow et al.; U.S. Pat. No. 6,517,864 to Orup Jacobsen et al.; U.S. Pat. No. 6,512,950 to Li et al.; U.S. Pat. No. 5,968,006 to Hofmann; and U.S. Pat. No. 5,749,847 to Zewart et al.

The technique of sonophoresis utilizes ultrasound to disrupting the stratum corneum, creating cavitations which disorder the lipid bilayers resulting increased drug transport. Although a variety of ultrasound conditions have been used for sonophoresis, the most commonly used conditions correspond to frequencies in the range of between one MHz and three MHz, and intensity in the range of between above zero and two W/cm 2 (U.S. Pat. No. 4,767,402 to Kost, et al.). Other devices use low frequency ultrasound that is less than one MHz (U.S. Pat. No. 6,234,990). Other examples of sonophoretic devices include but are not limited to those described in U.S. Pat. No. 6,491,657 to Rowe et al.; U.S. Pat. No. 6,487,447 to Weimann et al.; U.S. Pat. No. 6,190,315 to Kost et al.; U.S. Pat. No. 6,041,253 to Kost et al.; U.S. Pat. No. 5,947,921 to Johnson et al.; U.S. Pat. No. 5,906,580 to Kline-Schoder et al.; and U.S. Pat. No. 5,445,611 to Eppstein et al.

An additional method used to facilitate the transport of compounds across the stratum corneum is the use of thermal energy. Examples of the use of thermal energy technology to facilitate transport of compounds across the stratum corneum include but are not limited to those described in U.S. Pat. No. 6,780,426 to Zhang et al.; U.S. Pat. No. 6,613,350 to Zhang et al.; U.S. Pat. No. 6,465,006 to Zhang et al.; U.S. Pat. No. 6,284,266 to Zhang et al.; U.S. Pat. No. 6,261,595 to Stanley et al.; U.S. Pat. No. 6,048,337 to Svedman; U.S. Pat. No. 4,898,592 to Latzke et al.; U.S. Pat. No. 4,685,911 to Konno et al.; and U.S. Pat. No. 4,230,105 to Harwood.

Magnetophoresis, the use of magnetic energy, is an additional method used to increase drug transport across the stratum corneum. Some examples of magnetophoretic delivery devices include but are not limited to those disclosed in U.S. Pat. No. 6,564,093 to Ostrow et al.; U.S. Pat. No. 5,983,134 to Ostrow; U.S. Pat. No. 5,947,921 to Johnson et al.; U.S. Pat. No. 4,702,732 to Powers et al.

Microneedles or microstructured arrays are used to create micropores in the stratum corneum to aid in the flux of drugs across the skin. Examples of microneedle technology includes but is not limited to the disclosure in U.S. Pat. No. 6,331,310 to Roser et al. and H. Sebastien, et al, J. Pharm. Sci. 87:922-925 (1998).

Preservatives

In a preferred embodiment, the compositions of the invention further comprise a preservative. In general, topical formulations require preservation from microbial contamination that can effect the stability of the formulation and infect the user. When present in a composition of the invention, the amount of preservative is preferably from about 0.001% to about 1% by weight of the total composition weight, more preferably from about 0.01% to about 0.5% by weight. In some instances, It is also advantageous to include an antioxidant to preserve medicaments and excipients present in topical formulations. Some medicaments and excipients are oxygen labile and can undergo oxidation. When present in a composition of the invention, the amount of antioxidant is preferably from about 0.001% to about 1% by weight of the total composition weight, more preferably from about 0.01% to about 0.5% by weight.

Examples of preservatives include, but are not limited to, quaternary amines, such as quaternium 15, benzalkonium chloride, cetrimide, benzethonium chloride; and imidizolidinyl urea; organic acids, such as sorbic acid, p-hydroxybenzoic acid, and benzoic acid; parabens, such as methyl paraben and propyl paraben; alcohols, such as benzyl alcohol and isopropyl alcohol; phenols, such as triclosan, chlorhexidine, and thimerosal; hydantoin derivatives; chloromethylthiazoline; methylisothiazoline; phenyoxyethol; hexetidine; chlorohexydingluconate; and imidazolidinylurea. Preferably the preservative is methyl paraben, propyl paraben, or a mixture thereof.

Examples of antioxidants include, but are not limited to, ascorbic acid and its esters, sodium bisulfite, sodium metabisulfite, thiourea, butylated hydroxytoluene, butylated hydroxyanisole, tocopherols, alkyl gallates, scorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and chelating agents like EDTA and citric acid, and combinations thereof.

Anti-Foaming Agents

In a preferred embodiment, the compositions of the invention further comprise an anti-foaming agent to facilitate manufacture. Anti-foaming agents dissipate foam by destabilizing the air-liquid interface and allow liquid to drain away from air pockets. When present in a composition of the invention, the amount of anti-foaming agent is preferably from about 0.01% to about 1% by weight of the total composition weight, more preferably from about 0.1% to about 0.5% by weight.

Examples of anti-foaming agents include simethicone, dimethicone, ethanol, and ether.

Emollients, Humectants, and Skin Protectants

In a preferred embodiment, the compositions of the invention further comprise an emollient, a humectant, or a skin protectant, preferably a humectant to soothe and hydrate the skin. When present in a composition of the invention, the amount of humectant, skin protectant, or emollient is preferably from about 1% to about 10% by weight of the total composition weight, more preferably from about 2% to about 5% by weight.

Examples of humectants include, but are not limited to, glycerin, sorbitol, triacetin, polyethylene or butylenes glycols, urea, propylene glycol, 1,3-butylene glycol, ethanol, and isopropanol, and combinations thereof. In a preferred embodiment sorbitol is the humectant, preferably, 70% aqueous sorbitol solution. Examples of emollients include, but are not limited to, cholesterol and glycerol, myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and combinations thereof. Examples of skin protectants include, but are not limited to, vitamin E oil, allatoin, glycerin, zinc oxide, vitamins A, B (e.g. biotin and pantothenic acid), C, E, F, H, and P, and esters thereof.

Penetration Enhancers

In another embodiment, the compositions of the invention can further comprise a penetration enhancer. When present in a composition of the invention, the amount of penetration enhancer is preferably from about 1% to about 10% by weight of the total composition weight, more preferably from about 2% to about 5% by weight.

Penetration enhancers can be included in the compositions of the invention to optimize transfer of the NMDA-receptor antagonist through the stratum corneum and into the dermis/dermatome to provide a local effect. For a discussion of use of penetration enhancers in topical formulations see generally, Percutaneous Penetration Enhancers (Eric W. Smith & Howard I. Maibach eds. 1995); Ghosh, T. K. et al. 17 Pharm. Tech. 72 (1993); Ghosh, T. K. et al. 17 Pharm. Tech. 62 (1993); Ghosh, T. K. et al. 17 Pharm. Tech. 68 (1993), all of which citations are hereby incorporated herein by reference. The penetration enhancer should be pharmacologically inert, non-toxic, and non-allergenic, have rapid and reversible onset of action, and be compatible with the compositions of the invention.

Examples of penetration enhancers include, but are not limited to, transcutol P, ethyl alcohol, isopropyl alcohol, lauryl alcohol, salicylic acid, octolyphenylpolyethylene glycol, polyethylene glycol 400, propylene glycol, N-decylmethylsulfoxide, DMSO and the azacyclo compounds, as disclosed in U.S. Pat. Nos. 4,755,535; 4,801,586; 4,808,414; and 4,920,101, all of which patents are hereby expressly incorporated herein by reference. Preferably, the penetration enhancer is transcutol P.

Other Local Anesthetics

The compositions of the invention can further comprise one or more additional local anesthetics besides a NMDA-receptor antagonist in a topical formulation. As used herein, the term “local anesthetic” means any compound or composition that provides local numbness or analgesia or any drug that provides a regional blockage of nociceptive pathways (afferent and/or efferent). The local anesthetic can be any local anesthetic known or to be developed. When present in a composition of the invention, the amount of local anesthetic is preferably from about 0.1% to about 10% by weight of the total composition weight.

Examples of local anesthetics suitable for use with the invention include sodium channel blockers. Sodium channel blockers, such as lidocaine prevent the generation and conduction of nerve impulses by decreasing or preventing the large transient increase in the ermeability of excitable membranes to Na+. Examples of sodium channel blockers include, but are not limited to, ambucaine, amolanone, amylcaine, benoxinate, benzocaine, etoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutylp-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolarmine, or pharmaceutically-acceptable salts thereof, or mixtures thereof. Preferred sodium channel blockers, include lidocaine, procaine, bupivacaine, prilocalne, mepivacaine, etidocaine, ropivacaine, dibucaine, and pharmaceutically-acceptable salts thereof and mixtures thereof. The most preferred local anesthetic is lidocaine and pharmaceutically acceptable salts thereof.

Opioids, such as morphine are known to have local-anesthetic properties when topically administered in mammals. See, for example, U.S. Pat. No. 5,948,389 (issued Sep. 7, 1999) and Christoph Stein & Alexander Yassouridis 71 Pain 119 (1997).

As used herein the term “opioid” means all agonists and antagonists of opioid receptors, such as mu (μ), kappa (κ), and delta (δ) opioid receptors and subtypes thereof. For a discussion of opioid receptors and subtypes see Goodman & Gilman's the Pharmacological Basis of Therapeutics 521-525 (Joel G. Hardman et al. eds., 9th ed. 20 1996), hereby expressly incorporated herein by reference. The opioid can be any opioid receptor agonist or antagonist known or to be developed. Preferred opioids interact with the μ-opioid receptor, the K-opioid receptor, or both. Preferably, the opioid is an opioid-receptor agonist.

Examples of suitable opioids include, but are not limited to, alfentanil, allylprodine, alphaprodine, anileridine, benzylmorphine, benzitramide, nor-binaltorphimine, bremazocine, buprenorphine, butorphanol, clonitazene, codeine, CTOP, DAMGO, desomorphine, dextromoramide, dezocine, diampromide, dihydrocodeine, dihydrocodeine enol acetate, dihydromorphine, dimenoxadol, dimepheptanol, dimethylthiambutene, dioxaphetyl butyrate, dipipanone, diprenorphine, DPDPE, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levorphanol, lofentanil, loperamide, meperidine, meptazinol, metazocaine, methadone, metopon, morphine, myrophine, nalbuphine, naltrindole, benzoylhydrazone, naltrexone, narceine, nicomorphine, norlevorphanol, normethadone, nornorphine, norpipanone, opium, oxycodone, oxymorphone, papavereturn, papaverine, pentazocine, phenadoxone, phenazocine, phenoperidine, piminodine, pirtramide, proheptazine, promedol, propiram, propoxyphene, remifentanil, spiradoline, sufentanil, tilidine, U50,488, and U69,593, amiphenazole, cyclazocine, levallorphan, nalmefene, nalorphine, naloxone, and naltrexone or pharmaceutically-acceptable salts thereof, or mixtures thereof.

Examples of peptide opioids include, but are not limited to, Tyr-Gly-Gly-Phe-Leu ([Leu 5]enkephalin), Tyr-Gly-Gly-Phe-Met ([Met 5]enkephalin), Tyr-Gly-Gly-Phe-Leu-Arg-Arg-Ile-Arg-Pro-Lys-Leu-Lys-Trp-As p-Asn-Gln (DynorphinA), Tyr-Gly-Gly-Phe-Leu-Arg-Arg-Gln-Phe-Lys-Val-Val-Thr (Dynorphin B), Tyr-Gly-Gly-Phe-Leu-Arg-Lys-Tyr-Pro-Lys (α-Neoendorphin), Tyr-Gly-Gly-Phe-Leu-Arg-Lsy-Tyr-Pro (β-Neoendorphin), Tyr-Gly-Gly-Phe-Met-Thr-Ser-Glu-Lys-Ser-Gln-Thr-Pro-Leu-Val-Thr-Leu-Phe-Lys-Asn-Ala-Ila-Ile-Lsy-Asn-Ala-Tyr-Lys-Lys-Gly-Glu ((βh-Endorphin), [D-Ala 2, MePhe 4 Gly(ol) 5]enkephalin (DAMGO), [D-Pen 2, D-Pen 5]enkephalin (DPDPE), [D-Ser 2, Leu 5]enkephalin-Thr 6 (DSLET), [D-Ala 2, D-Leu 5]enkephalin (DADL), D-Phe-Cys-Tyr-D-Trp-Orn-Thr-Pen-Thr-NH 2 (CTOP), [D-Ala 2, N-MePhe 4, Met(O) 5-ol]enkephalin (FK-33824), Tyr-D-Ala-Phe-Asp-Val-Val-Gly-NH 2 ([D-Ala 2]Deltorphin 1), Tyr-D-Ala-Phe-Glu-Val-Val-Gly-NH 2 ([D-Ala 2 Glu 4]Deltorphin (Deltorphin II)), Tyr-Pro-Phe-Pro-NH 2 (Morphiceptin), Tyr-Pro-MePhe-D-Pro-NH 2 (PL017), [D-Ala 2, Leu 5, Cys 6]enkephalin (DALCE) or pharmaceutically-acceptable salts thereof, or mixtures thereof. Preferred opioids include morphine, loperamide, and loperamide derivatives such as those disclosed in U.S. Pat. Nos. 5,763,445; 5,981,513; 5,869,521; 5,744,458; 5,760,023; 5,798,093; 5,849,762; 5,811,078; 6,004,964; 5,962,477; 5,688,955; 5,888,494; 5,646,151; and 5,667,773 or pharmaceutically-acceptable salts thereof, or mixtures thereof, all of which patents are hereby expressly incorporated herein by reference. The most preferred opioid is morphine or a pharmaceutically-acceptable salt thereof.

Other agents with local-anesthetic properties include analgesics, such as nonsteroidal anti-inflammatories (“NSAIDs”), see, for example, Transdermal and Topical Drug Delivery Systems 87-93 (Tapash K. Ghosh et al. eds., 1997). Examples of non-narcotic analgesics with local-aesthetic properties include, but are not limited to, acetylsalicylic acid, ketoprofen, piroxicam, diclofenac, indomethacin, and ketorolac.

In yet another embodiment of the current invention, agents may be included in the compositions of the invention to prolong the local-anesthetic effect, such as, a glucocorticosteroid (see, for example, U.S. Pat. No. 5,922,340, incorporated herein by reference) or a vasoconstrictor, such as a catecolamine.

Other Excipients

The compositions of the invention can further comprise one or more additional ingredients, such as one or more thickening agents, medicinal agents or pharmaceuticals, bioadhesive polymers, inert carriers, lipid absorbents, viscosity stabilizers, chelating agents, buffers, anti-fading agents, stabilizers, moisture absorbents, fragrances, colorants, film-forming materials, and refatting agents, etc. One of skill in the art will readily be able to choose such additional excipients based on the physical and chemical properties desired in the final topical formulation. Of course, a single excipient may have multiple functions and properties.

Thickening Agents

The compositions of the invention can further comprise one or more thickening agents. Thickening agents are used to increase viscosity and improve bioadhesive properties. When present in a composition of the invention, the amount of thickening agent is preferably from about 1% to 10% by weight of the total composition weight, more preferably from about 2% to about 5% by weight.

Examples of thickening agents include, but are not limited to, cellulose, hydroxypropyl cellulose, methyl cellulose, polyethylene glycol, sodium carboxymethyl cellulose, polyethylene oxide, xanthan gum, guar gum, agar, carrageenan gum, gelatin, karaya, pectin, and locust-bean gum, aliginic acid, bentonite carbomer, povidone, and tragacanth.

Medicinal Agents

The compositions of the invention can include medicinal agents or their pharmaceutically acceptable salts. One of skill in the art can readily choose a medical agent to incorporate into the compositions of the invention and its appropriate concentration depending on the indication and desired effect. Examples of medicinal agents include, but not limited to, antifungals such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics, such as mupirocin, erthromycin, clindamycin, gentamicin, polymyxin, bacitracin, and silver sulfadiazine; antiseptics, such as iodine, povidine-iodine, benzalkonium chloride, benzoic acid, chlorhexidine, nitrofurazone, benzoyl peroxide, hydrogen peroxide, hexachlorophene, phenol, resorcinol, and cetylpyridinium chloride; and anti-inflammatories, such as hydrocortisone, prednisone, triamcilolone, betamethasone, dexamethasone.

Bioadhesive Polymers

The compositions of the invention can include one or more bioadhesive polymers. Bioadhesive polymers are also useful in the present invention to hydrate the skin and enhance its permeability. Bioadhesive polymers can also function as thickening agents. Examples of bioadhesive polymers include, but are not limited to, pectin, alginic acid, chitosan, hyaluronic acid, polysorbates, such as polysorbate-20, -21, -40, -60, -61, -65, -80, -81, -85; poly(ethyleneglycol), such as PEG-7, -14, -16, -18, -55, -90, -100-135, -180, -4, -240, -6, -8, -9, -10, -12, -20, or -32; oligosaccharides and polysaccharides, such as gellan, carrageenan, xanthan gum, gum Arabic, and dextran; cellulose esters and cellulose ethers; modified cellulose polymers, such as carboxymethylcellulose, hydroxyethylcellulose, hydroxypropyl methylcellulose, hydroxyethyl ethylcellulose; polyether polymers and oligomers, such as polyoxyethylene; condensation products of poly(ethyleneoxide) with various reactive hydrogen containing compounds having long hydrophobic chains (e.g. aliphatic chains of about 12 to 20 carbon atoms), for example, condensation products of poly(ethylene oxide) with fatty acids, fatty alcohols, fatty amides, polyhydric alcohols; polyether compounds, such as poly(methyl vinyl ether), polyoxypropylene of less than 10 repeating units; polyether compounds, such as block copolymers of ethylene oxide and propylene oxide; mixtures of block copolymers of ethylene oxide and propylene oxide with other excipients, for example, pluronic lethicin organogel (see 1 International Journal of Pharmaceutical Compounding 71 (1997)); poly(vinyl alcohol); polyacrylamide; hydrolyzed polyacrylamide; poly(vinyl pyrrolidone); poly(methacrylic acid); poly(acrylic acid) or crosslinked polyacrylic acid, such as carbomer, i.e., a homopolymer of acrylic acid crosslinked with either an allyl ether of pentaerythritol, an allyl ether of sucrose, or an allyl ether of propylene (e.g., Acrisint® 400, 410, or 430 commercially available from 3V Inc. Weehawkin, N.J.); Orabase® (i.e., a mixture of gelatine, pectin and sodium carboxymethyl cellulose in a plasticized hydrocarbon gel, commercially available from Hoyt laboratories, Needhm, Me.); Carafate® (sulfated sucrose and aluminum hydroxide, commercially available from Marion Laboratories, Inc., Kansas City, Mo.). The block copolymers of ethylene oxide and propylene oxide are particularly preferred

Methods of Manufacture

The compositions of the invention is prepared according to standard methods, well known in the art, for preparing emulsions for topical administration. For example, the methods recited in Gennaro, A. R., Remington: The Science and Practice of Pharmacy, 21st edition, Lippincott, Williams & Wilkins (2006), hereby expressly incorporated herein by reference, can be used. Also, Example preparations are recited in the Example section below.

The compositions described herein can be made by cold compounding. This is significant since one or more of the compounds admixed in the topical compositions described herein may be sensitive to heat or other types of energy. Thus the activity of the composition may be detrimentally affected as a result of the formulation of the compositions in other manners. Preferably, the ingredients of this topical composition can be merely mixed together, without heating and using a sufficient amount of the carrier to provide a substantially homogeneous cream or gel. It is generally preferred to dissolve, disperse or suspend one or more of the ingredients prior to cold compounding in order to ensure substantially homogeneous distribution of the active ingredients in the composition.

Alternatively, the components can be separated into those that are water-soluble and those that are oil-soluble. The water-soluble components can be mixed together in one vessel to form a solution and the oil-soluble components can be mixed together in a separate vessel and heated (e.g., 70° C. to 80° C.) to form a solution. The two solutions can then be mixed and the mixture allowed to cool. This method requires nothing more than two beakers and a heating apparatus. Homogenation is achieved using a high-shear rate blender or other suitable apparatus. The appropriate droplet size is achieved by standard adjustment of the shear rate during high-speed mixing followed by droplet size analysis as described in Gennaro, A. R., Remington: The Science and Practice of Pharmacy, 21st edition, Lippincott, Williams & Wilkins (2006) and Allen & Terence, Particle Size Measurement 483 (4th ed. 1990, both or which citations are hereby expressly incorporated herein by reference. Suitable equipment and methods for preparing emulsions and compositions of the invention, such as high-shear rate blenders are described in 2 Remington: The Science and Practice of Pharmacy 1509-1515 (Alfonso R. Gennaro ed., 19th ed. 1995) (updated in Gennaro, A. R., Remington: The Science and Practice of Pharmacy, 21st edition, Lippincott, Williams & Wilkins (2006)), hereby expressly incorporated herein by reference. Methods for preparation of emulsions for topical administration, suitable for preparing compositions of the invention, are also described in Bernard Idson, Pharmaceutical Emulsions in 1 Pharmaceutical Dosage Forms: Disperse Systems 199 (Herbert A. Lieberman et al. eds. 1988), hereby expressly incorporated herein by reference.

The compositions of the invention are then packaged and stored according to well-known methods. For example, see the packaging procedures described in 1 Remington: The Science and Practice of Pharmacy 390-391 (Alfonso R. Gennaro ed., 19th ed. 1995—updated in Gennaro, A. R., Remington: The Science and Practice of Pharmacy, 21st edition, Lippincott, Williams & Wilkins (2006)), hereby incorporated herein by reference. If desired, the compositions of the invention can be sterilized according to well-known methods, for example, the methods described in 2 Remington: The Science and Practice of Pharmacy 1463-1486 (Alfonso R. Gennaro ed., 19th ed. 1995—updated in Gennaro, A. R., Remington: The Science and Practice of Pharmacy, 21st edition, Lippincott, Williams & Wilkins (2006)), hereby incorporated herein by reference.

The present invention and its many attendant advantages will be understood from the foregoing description and it will be apparent that various changes in form, construction and arrangement of the parts thereof may be made without departing from the spirit and scope of the invention or sacrificing all of its material advantages, the form hereinbefore described are merely exemplary embodiments thereof.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 15% to about 30%” should be interpreted to include not only the explicitly recited values of about 15% to about 30%, but also include individual values and subranges within the indicated range. Thus, included in this numerical range are individual values such as 15, 16, 17, 18, 19, 20, 21, 22, 23, 24, 25, 26, 27, 28, 29, and 30, and sub-ranges such as from 15 to 25, 20 to 25, and from 20 to about 30, etc. This same principle applies to ranges reciting only one numerical value. Furthermore, such an interpretation should apply regardless of the breadth of the range or the characteristics being described.

Application

Subjects to whom the formulations can be administered are primarily mammals, including humans, pets, and livestock and other farm animals and sport animals. The compositions and methods described herein are preferably used on but not limited to humans, but may also include pets, such as dogs and cats; farm mammals, such as horses, cows, pigs, and sheep; and laboratory animals, such as monkeys, guinea pigs, rats, rabbits, and mice.

The site of application is dependent on many factors including, but not limited to, the amount of drug to be delivered, the extent of enhancement required, the side effects manifested and the time of application. Thus, another important facet of the methods described herein is the use of these compositions, alone or in combination with other drugs, or to apply such formulations, or topical products in general, specifically to the soles of the feet, the palms of the hands or other immune-privileged sites of the body. Also, the drugs, compositions or products may be administered later in the day or at night when the permeability at the site of application is higher.

The general mode of action of the composition is through “topical administration.” The term “topical administration” or “topical application” refers to directly layering or spreading upon epidermal tissue, especially outer skin or membrane, including but not limited to the skin or membrane of cutaneous, mucosal or oral, vaginal, rectal, ocular, or nasal surfaces or cavities. The composition is topically administered to a subject in an amount and duration sufficient to prevent or relieve pain associated with any cause, including, but not limited to, neuropathic inflammation, and acute and chronic peripheral neuropathy.

Methods described herein can also involve the topical application of a composition described herein to areas of the skin in the vicinity of tissue that suffers from neuropathic pain. In particular, the compositions and methods described herein are useful on the patients' extremities such as the peripheral appendages (e.g., fingers, toes, hands, arms, leg, and feet) and general areas of pain (e.g., torso, back, shoulder, neck, head) where the neuropathic pain, particularly peripheral neuropathy, is often the most pervasive, or the dermatome site along the spine. The site can also be in the vicinity of tissue that has undergone traumatic injury such as surgery, amputation, lesion, infection or other such injury. The methods and compositions described herein can also be applied to the specific ganglia that mediate pain to the spinal column and to the spine itself. Specific dermatomes are involved for the correct application of the compositions described herein for neuropathic analgesia.

Administration to the subject is performed in accordance with that mode which is most amenable to the topically acceptable form chosen. For example, gels, lotions, creams, and ointments are preferably administered by spreading. Because hydrated skin is more permeable than dry skin, the dosage form can be modified or an occlusive dressing can be applied to facilitate absorption. Also contemplated by the compositions and methods described herein are slow-release or sustained-release forms, whereby a relatively consistent level of the composition, particularly the NMDA antagonist is provided over an extended period.

The compositions of the invention can be topically administered to intact skin by a medical professional or by the patient by simple mechanical rubbing into the application site, or by applying a transdermal patch to the site. In applying these compositions to the skin, for maximum effectiveness and increased absorption, the area to which the composition is administered is covered with a hot, dampened cloth for approximately one minute. The area is then allowed to dry for a few seconds. Next, the composition of the invention is rubbed on to the complete target area of the skin (the painful area) and gently, but firmly, massaged in with the fingertips until all visible gel or cream has been absorbed.

The surface area that is covered by the topical composition following application must be sufficient to provide for the desired amount of agent administration, and in representative embodiments ranges from about 1 to 200 cm², and in many embodiments from about 10 to 180 cm², usually from about 10 to 100 cm², e.g., 10, 20, 30, 40 or 50 cm². For example, in the case of diabetic neuropathy, the subject may apply the invention topical treatment over the entire foot and lower leg, or the arm/forearm. In representative embodiments, the period of time that the composition is maintained at the site of application does not exceed about 48 hours, and in representative embodiments does not exceed about 24 hours. However, the period of time during which the preparation is maintained at the application site is, in representative embodiments, at least about 15 to 30 minutes, usually at least about 1 hour. In practicing the subject methods, a given dosage of the topical composition may be applied a single time or a plurality of times over a given time period, e.g., the course of the pain condition being treated, where the dosing schedule when a plurality of compositions are administered over a given time period may be daily, weekly, biweekly, monthly, etc. Treatment can be applied as needed and for such length as determined by the healthcare provider, e.g., physician, or on the level of pain.

For example, a suitable amount of a composition described herein can be applied one to six times daily as needed to relieve pain and other symptoms of neuropathy. Preferably, the composition is applied two to four times daily, as needed for pain. A sufficient amount is applied to cover the area afflicted by the neuropathy with a thin layer of the composition and the composition is rubbed into the skin until little or no residue remains on the skin. Treatment begins initially to treat acute symptoms but may be continued indefinitely to relieve pain, prevent symptoms of neuropathy from returning and possibly restore some nerve and/or skin function. The application frequency and volume of the composition may decrease over time, but not necessarily. With gels, creams, or ointments, typically 1 to 10 applications are required per day, more preferably 2, 3 or 4 applications per day, and even more preferably as needed. Generally, the greater the level of pain the greater the number of applications.

The methods described herein also encompass topical administration of compositions in a physiologically acceptable topical vehicle and in an amount and duration sufficient to provide an antineuropathic response. Hence the terms “transdermal”, “topical” and “transmucosal” are used interchangeably unless specifically stated otherwise. Likewise the terms “skin,” “derma,” “epidermis,” “mucosa,” and the like shall also be used interchangeably unless specifically stated otherwise.

A specific mode of administration of the compositions described herein is through “topical administration.” As used herein, “transdermal” or “percutaneous” delivery relates to delivery of a drug by passage into and through the skin or mucosal tissue. This mode of action is restricted to the region of the dermis where the drug application has occurred. In using the topical route of administration, the amount of composition absorbed systemically is generally minimal. The vehicle can, however, allow the active ingredients to efficiently penetrate tissues when applied topically and can allow increased concentrations of particular components (e.g., ketamine) and all added agents in the compositions described herein. Topical administration of the compositions described herein is directed to cutaneous surfaces. The composition can be applied topically on a subject in an amount and duration sufficient to prevent or relieve pain associated with any cause, including, but not limited to, neuropathic inflammation, and acute and chronic peripheral neuropathy.

A subject can be treated in accordance with the compositions described herein by administering the composition suspended in or admixed with a physiologically suitable topical vehicle and manually applied or sprayed (either with a manually-actuated pump or with the aid of a suitable pharmaceutically-acceptable propellant) onto the surface area in need of treatment. Preferably, the composition is applied by topical massage. Suitable formulations for topical application of drugs are well known to those of ordinary skill in the art and can be routinely selected.

The amount of composition to be applied varies on the choice of vehicle as well. For example, when the composition is administered by spraying an alcoholic liquid solution of the drug, the total volume in a single dose can be very low. Conversely, when the compositions described herein are administered in a topical cream, the total volume can be higher. The vehicle selected and its manner of application is preferably chosen in consideration of the needs of the patient and the preferences of the administering medical practitioner.

In one embodiment of the current invention, the compositions of the invention are contained in a patch that is applied adjacent to the area of skin to be treated. As used herein a “patch” comprises at least a composition of the invention and a covering layer, such that, the patch can be placed over the area of skin to be treated. Preferably, the patch is designed to maximize drug delivery through the stratum corneum and into the epidermis or dermis, and to minimize absorption into the circulatory system, reduce lag time, promote uniform absorption, and reduce mechanical rub-off.

Examples of patches suitable for use with compositions of the invention include (1) the matrix-type patch; (2) the reservoir-type patch; (3) the multi-laminate drug-in-adhesive type patch; (4) the monolithic drug-in-adhesive type patch; and (5) hydrogel patch; see generally Ghosh, T. K.; Pfister, W. R.; Yum, S. I. Transdermal and Topical Drug Delivery Systems, Interpharm Press, Inc. p. 249-297, hereby expressly incorporated herein by reference). These patches are well known in the art and available commercially.

In general, the active ingredient (e.g., NMDA antagonist) of the invention will comprise from about 0.5 percent to about 40 percent by weight of the patch, preferably from about 10 percent to about 30 percent, more preferably from about 15 percent to about 25 percent, and most preferably from about 18 percent to about 22 percent by weight of the patch.

The patches for use with compositions of the invention can be manufactured, packaged, stored and labeled according to standard procedures. For example, see the procedures described in Bova et al., Product Development and Technology Transfer for Transdermal Therapeutic Systems in Transdermal Controlled Systemic Medications 379-396 (Y. W. Chien ed. 1987); J. W. Dohner, Development of Processes and Equipment for Rate Controlled Transdermal Therapeutic Systems in Transdermal Controlled Systemic Medications 349-364 (Y. W. Chien ed. 1987); H-M Wolf et al., Development of Processes and Technology for Adhesive-Type Transdermal Therapeutic Systems in Transdermal Controlled Systemic Medications 365-378 (Y. W. Chien ed. 1987), all of which citations are hereby incorporated herein by reference.

Topical or transdermal application of the compositions described herein is useful for relieving pain, inflammation and irritation associated with skin diseases and disorders. Painful lesions develop, for example, from viral infections, i.e., herpes zoster, skin cancers and genetic disorders. Acute post-operative or surgical pain can be reduced or even eliminated as can pain associated with chronic disorders, such as diabetic peripheral polyneuropathy. The methods described herein may also provide one or more of the beneficial effects described above. In addition, methods described herein can provide some additional beneficial effects due to one or more of the ingredients contained in the pharmaceutically acceptable carrier such as described above, e.g., the return of sensory perception at the application site.

Dosage

The amount of composition necessary to produce a therapeutic effect at an affected area can be based on various factors, including the strength of the active ingredients, the ingredients admixed, the pain type and intensity, or related to, the location and size of the area and the relative condition that is to be treated, side effect profiles, or can be based on factors targeting consensus or generalized populations. For example, the amount of composition needed to treat severe pain is likely to be greater than the amount of composition needed to treat mild to moderate forms of the affliction. In addition, an acute condition will likely require medication for less time or shorter duration than a chronic condition, or alternatively, lesser frequency of application. Individual sensitivities will also influence the dosage amounts administered to a particular subject. A determination of the appropriate dose is within the skill of one in the art given the parameters herein. In terms of the compositions described herein, the dosage range is preferably determined by considering the amount of ketamine in in percentage, and the surface area to be treated. The concentration of the active ingredients in the pharmaceutical composition can be from about 0.001% to about 50% NMDA antagonist (e.g., ketamine) of the total composition. Additional compounds, such as those listed above that reduce or may reduce NMDA antagonist neurotoxicity, can be added from about 0.001% to about 50% of the total composition. In accordance with the compositions and methods described herein, the foregoing doses can be readily optimized following the teachings herein, based on known pharmacological protocol, by those of ordinary skill in the art, with no more than routine optimization. Of course, the preferred lower limit for drug delivery is that necessary to bring about an anti-neuropathic effect. The preferred upper limit is less than that amount which produces untoward side effects.

Although not crucial, the dilution and/or formulation of the active ingredients of the compositions described herein, in a physiologically acceptable topical vehicle, can be important and useful in providing the final dosage concentration. The compositions can be supplied in solid, semi-solid or liquid forms, including tablets, capsules, powders, liquids, and suspensions. The compositions described herein can therefore encompass concentrated forms for subsequent dilution before use or sale. The compositions can comprise any physiologically acceptable topical excipients including, but not limited to, gels, lotions, creams, ointments, and liquids, as further elaborated herein.

Selection of the appropriate dosage of the invention composition for the application site is an important consideration. The rate of topical analgesic administration from the topical formulation or patch is a function of skin permeability, and skin permeability has been shown to vary between anatomical sites depending on the thickness of the stratum corneum. For example, the permeability, in general, increases in order from planter foot arch, lateral ankle, palm, ventral forearm, dorsal forearm, back, chest, thigh, abdomen, scalp, axilla, forehead, and scrotum; see R. C. Wester. & H. I. Maibach Regional variation in Percutaneous Absorption in Percutaneous Absorption, Mechanism, Methodology, Drug Delivery 111-119 (R. L. Bronaugh & H. I. Maibach eds., 2nd ed. 1989), hereby expressly incorporated herein by reference. Of course, the dosages and dosing frequency will be determined by a trained medical professional and will depend upon many factors such as application site and size and the severity of the indication.

In general, a dosage from about 0.05 or 0.1 mg/kg to about 5 g/kg subject body weight may be utilized to carry out the present invention, more preferably from about 1 mg/kg to about 1 mg/kg per application. Preferably, approximately 0.5 g to about 2 g of invention topical preparation is applied per administration, with about 10-30% of the preparation being the NMDA antagonist. Generally, about 0.1 g/cm² of skin area to about 5 g/cm², preferably 0.1 mg/cm² to about 2 g/cm² of a composition of the invention is administered to and around the application site. More preferably the dosage form will be from 0.1 mg/cm² to about 1 g/cm², more preferably 0.5 mg/cm² to about 0.5 g/cm² per application.

The therapeutically effective dosage of any specific compound, the use of which is in the scope of present invention, will vary somewhat from compound to compound, and patient to patient, and will depend upon the condition of the patient and the route of delivery. Exemplary duration of the treatment may be one to ten dosages per day for a period of one to several days, one to several weeks, such as two to three weeks, one or several months, or until the condition is controlled or treated. In some embodiments lower doses given less frequently can be used to prevent or reduce the incidence of recurrence of the condition being treated.

When a patch is used to administer a composition of the invention, the dosage to achieve pain relief is determined by the active surface area of the medicated portion of the patch in direct contact with the skin. Several dosage strengths are advantageous depending upon the severity of the wound. In general, a physician can begin dosing with a low or intermediate strength patch and then, depending upon the effectiveness, adjust the dosage up or down by prescribing a patch of higher or lower active concentration or a patch of larger or smaller surface area, or, in some cases, multiple patches. In general, the composition of the invention will comprise from about 0.5 percent to about 20 percent by weight of the patch, preferably from about 5 percent to about 25 percent by weight of the patch. For matrix (drug-in-adhesive) type patches, the compositions of the invention will comprise from about 0.5 percent to about 20 percent by weight of the patch. For patches comprising a hydrogel, the compositions of the invention will comprise from about 0.5 percent to about 10 percent by weight of the patch. Fresh patches may be administered multiple times per day, but, preferably, a fresh patch is administered about every 18 to about every 48 hours, more preferably daily.

### EXAMPLES

All chemicals used in the following examples are available from commercial sources in the United States of America for instance, Hawkins Pharmaceuticals (Minneapolis, Minn.) or B&B Pharmaceuticals (Aurora, Colo.). The preferred topical base “Lipoderm” is only available from the Professional Compounding Centers of America (PCCA). However, other topical bases are available from Hawkins Pharmaceuticals (e.g., “Lipo Cream”) or from Medisca, Inc. (Plattsburg, N.Y.). “Krisgel” (a thickener) is available only from PCCA, but similar commercial products are available from other chemical suppliers, e.g., “Tommy Gel” from Hawkins Pharmaceuticals. All reagents used in the Examples below are also commercially available from international standard sources, for example from, Spectrum Laboratory Products, Inc. Gardena, Calif.; Lab Express International Inc. NJ; AK Chemical Tech and Shandong Zhonggong Chemical Co. Ltd., Shanghai Gupeng International Trading Co., Ltd., Beijing Medicine Chemical Co., Ltd., in China; Greenspharma, Maps Pharmaceuticals of India. Additional searches online will result in additional sources for such compounds. One source for ketamine hydrochloride is from Medisca, Inc., Plattsburg, N.Y.

Transdermal bases differ from topical bases, e.g., petrolatum or cold cream, in that they limit the penetration of the active chemicals through all dermal layers. This subcutaneous entry, in turn, allows the active chemicals to penetrate the nerve fibers themselves. The PCCA transdermal base Lipoderm is preferred due to its proven penetration superiority over PLO (pluronic-lecithin-organogel), although PLO or other such compounds are contemplated by the present invention.

The three examples listed below are compositions according to the invention previously noted.

Formulation

Ketamine HCl powder is accurately weighed by any FDA-approved scale. Water is measured using any approved cylindrical graduate. The powders are first filtered through a fine-mesh screen into a glass mortar then dissolved by the addition of water. An electronic mortar and pestle (EMP) is equally suitable in place of a manual glass mortar and pestle. The Lipoderm (or similar) transdermal base is then geometrically levigated into the dissolved powders. Krisgel (or similar) is then stirred into the mixture until evenly distributed. The mixture is then milled in a three-roller ointment mill (Exakt 50 or similar) and then dispensed in an appropriate ointment jar.

Ketamine 20% - 100 g example size

| Component | Weight | | Weight % |
|---|---|---|---|
| Ketamine HCl | 20 | g | 20% |
| Water (purified) | 13 | ml | 13% |
| Lipoderm Base | 64 | g | 64% |
| Krisgel | 3 | ml | 3% |

Ketamine 25% - 100 g example size

| Component | Weight | | Weight % |
|---|---|---|---|
| Ketamine HCl | 25 | g | 25% |
| Water (purified) | 12 | ml | 12% |
| Lipoderm Base | 60 | g | 60% |
| Krisgel | 3 | ml | 3% |

Ketamine 30% - 100 g example size

| Component | Weight | Weight % |
|---|---|---|
| Ketamine HCl | 30 g | 30% |
| Water (purified) | 11.5 ml | 11.5% |
| Lipoderm Base | 56 g | 56% |
| Krisgel | 2.5 ml | 2.5% |

The anticipated dose of local ketamine can range from 0.5 gm to 5 gm per application up to 6 times daily, but typically about 1 gram per application, 2-5 times per day. This dose translates to a topical administration of 200 mg of ketamine hydrochloride per application.

The anticipated dose of local ketamine can range from 0.5 gm to 5 gm per application up to 6 times daily, but typically about 1 gram per application, 2-5 times per day. This dose translates to a topical administration of 200 mg of ketamine hydrochloride per application.

The anticipated dose of local ketamine can range from 0.5 gm to 5 gm per application up to 6 times daily, but typically about 1 gram per application, 2-5 times per day. This dose translates to a topical administration of 200 mg of ketamine hydrochloride per application.

Administration

The compositions described herein may be applied two ways via massage: (1) directly to the pain site or appropriate ganglion and (2) into the appropriate dermatome on the spine.

Plan 1 is normally used first, especially if the pain locus is below the patient's waistline (due to reduced systemic circulation of the agents.) The patient is instructed to find the most precise area of pain—if possible—by using a blunt, pointed object (i.e., fingertip, pen tip, etc.) By use of a “checkerboard pattern” search, many times the pain locus is discovered. For example, a foot pain locus may be found by pressing a fingertip on one side of the ankle for approximately 2 seconds then moving the fingertip an inch towards the other side of the ankle. This pressure is repeated “checkerboard style” (across and downward) until the entire foot—top and bottom—has been covered. The patient takes note of what area(s) hurt most and then treats the area(s) with ½ gram or 1 gram of cream at each pain site. If a precise locus cannot be found, then a 1 gram dose to the ganglion located ¾ inch below and ¾ inch behind the inside anklebone will suffice. This ganglion is responsible for innervation of the foot via the L-4, L-5, S-1, and S-2 dermatomes. Other ganglia may be used similarly for pain loci at other anatomical sites. An anesthesiologist—or a medical professional with a thorough understanding of human anatomy—should be consulted for the most appropriate ganglion (or ganglia) to be used.

Plan 2 is used when there is insufficient analgesia provided by Plan 1. Plan 2 requires massage of the cream into the appropriate dermatome on the spinal column. The patient is shown where the correct dermatome application site (on the spine) is for the painful area described by the patient. For example, a foot pain locus requires cream application to the L-4, L-5, S-1, and S-2 vertebrae on the spine.

How much cream to apply depends on (1) the pain site and (2) pain severity. The patient is instructed to use Plan 1 first. During the counseling session, the patient learns to (1) find the pain using the “checkerboard technique” described above and (2) prepare the skin for application by warming the site with a very warm, slightly moist cloth. A minimum dose—usually between ½ to 1 gram—is suggested as a starting dose. A (1-gram+½ gram) dosing spoon is given to the patient for accurate measure. The patient is instructed to use this starting dose 3 times daily for 3 days unless side effects appear. If that happens, the patient is counseled to immediately cease the applications and call his/her doctor. After the 3 day period—and if no sign of analgesia nor side effects—the dose may be increased by ½ gram increments daily. For example, if a 1-gram dose to the site did not relieve the pain during the first 3 days, then the dose would be increased by ½ gram per application on day 4. If the pain was still not managed, the dose would be increased by another ½ gram dose on day 5. The dose total at that point would be 2 grams per application. This sequence would be repeated until (1) the pain is managed or (2) side effects begin. Note: Side effects at any time are the limiting factor for dosing.

If Plan 1 does not provide sufficient analgesia within 7 days of the first application, then the patient is instructed to initiate Plan 2. Application to this area is explained above. Because the area is above the belt line, the patient is told that there is an increased risk of side effects. A 1-gram dose at the correct dermatome is started with the proviso that the dose may be adjusted down or up after a 3 day dosing period. This is similar to Plan 1.

Dosing frequency is dependent on the cream's duration of action. Duration of action varies from patient to patient. Normally, the cream is applied 3 times daily, but more frequent—or less frequent—applications are possible. Again, the limiting factor is side effects. Hence, if no side effects, then multiple daily applications are OK. The cream is a pain management “tool”. As such, the cream may be used as often as necessary (subject to side effects.)

Limited Ketamine Absorption

Blood was drawn from the arm of Subject 1 prior to any preparation or administration as control. Approximately 1 gram of topical formulation comprises of 25% ketamine in Lipoderm transdermal vehicle (25% ketamine in 1 gram total weight topical cream) is administered four separate applications throughout one day. 6 hours after final application, the forearm of Subject 1 was cleaned and prepared with wet warm towels. After 1 minute of preparation, the topical formulation is massaged on the entire forearm until the topical cream is entirely lathered on the forearm. 5 mL blood is then drawn approximately every 15-16 minutes for six hours. 5 mL blood is further drawn every six hours for 24 hours (e.g., hour 12, 18 and 24. Detection of any trace ketamine and norketamine is analyzed for using gas chromatography/mass spectrometry at very low concentrations (0.002 mg/ml). No systemic ketamine or norketamine is found in any of the samples. Continuous use of the compound does not affect Subject 1 as Subject 1 does not experience any side effects associated with ketamine such as dizziness, disorientation or hallucinations.

Pain Reduction

A survey was provided to over 800 patients provided with various ketamine topical formulations combined with other compounds, using PLO or Lipoderm as the transdermal base. For all diagnoses, greater analgesia was achieved with ketamine concentrations greater than or equal to 15%, with best results from 20% or greater. Results show that the various topical formulations were efficacious in relieving pain in 643 of 824 diagnoses (78%). 181 of these 824 diagnoses (22%) were treatment failures due to insufficient analgesia or side effects. The patients were treated for diabetic peripheral neuropathy, low back pain, polyneuropathy in the hands and feet, post-herpetic neuralgia, sciatica, CRPS/RSD, post-surgical neuropathy, and miscellaneous neuropathies including fibromyalgia.

Objectives and Advantages

Pain management is one objective of the compositions and methods described herein. The methods and compositions described herein can ameliorate neuropathic pain in patients. The compositions and methods described herein have the following advantages: (1) the compositions described herein are effective against a wide variety of sympathetically mediated pain (SMP) sources—including various neuropathies, low back pain, sciatica, and post-spinal surgery pain; (2) the doses needed to control neuropathic pain are relatively small; (3) dose volumes are also small—a distinct application advantage; (4) local analgesia with limited systemic ketamine minimizes patients affected by side effects; (5) the compositions described herein are cosmetically elegant; (6) the compositions described herein are easy to apply because they are readily absorbed by the prepared skin.

All cited references including publications and patent documents cited in this specification are herein incorporated by reference in their entireties as if each individual publication or patent application were specifically and individually indicated to be incorporated by reference. Although the foregoing methods and compositions have been described in some detail by way of illustration and example for purposes of clarity of understanding, it will be readily apparent to those of ordinary skill in the art in light of the teachings of these methods and compositions that certain changes and modifications may be made thereto without departing from the spirit or scope of the appended claims. The present invention is not to be limited in scope by the specific embodiments disclosed in the examples, which are intended as illustrations of a few aspects of the invention, and any embodiments that are functionally equivalent are within the scope of this invention. Indeed, various modifications of the invention in addition to those shown and described herein will become apparent to those skilled in the art and are intended to fall within the scope of the appended claims.

We claim:

1) A treatment for neuropathy comprising a therapeutically effective amounts of a ketamine formulated in a pharmaceutical topical vehicle that limits systemic penetration of ketamine.

2) The treatment of claim 1, wherein ketamine is limited particularly to the epidermis, dermis and the dermatome.

3) The treatment of claim 1, wherein the topical vehicle is designed to maximize ketamine delivery through the stratum corneum and into the epidermis or dermis or dermatome.

4) The treatment of claim 1, wherein topical delivery minimizes absorption into the circulatory system.

5) The treatment of claim 1, wherein the transdermal vehicle is a skin retardant.

6) The treatment of claim 1, wherein the neuropathy is selected from peripheral neuropathy, phantom limb pain, reflex-sympathetic dystrophy, causalgia, syringomyelia, and painful scar; specific neuralgias at any location of the body; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; and connective tissue disorders.

7) The treatment of claim 5, further comprising magnesium salt.

8) The treatment of claim 1, wherein ketamine is in an amount of greater than 10% by weight.

9) The treatment of claim 1, wherein ketamine is in an amount of equal or greater than 15% by weight.

10) The treatment of claim 1, wherein ketamine is in an amount of greater than 15% by weight.

11) The treatment of claim 1, wherein ketamine is in an amount of equal or greater than 20% by weight.

12) The treatment of claim 1, wherein ketamine is in an amount of equal or greater than 25% by weight.

13) A treatment for neuropathy comprising a therapeutically effective amount of an NMDA antagonist formulated in a pharmaceutical topical vehicle that limits systemic penetration of ketamine.

14) The treatment of claim 13, wherein the topical vehicle is designed to maximize ketamine delivery through the stratum corneum and into the epidermis or dermis or dermatome.

15) The treatment of claim 13, wherein the transdermal vehicle is a skin retardant.

16) The treatment of claim 13, wherein the neuropathy is selected from peripheral neuropathy, phantom limb pain, reflex-sympathetic dystrophy, causalgia, syringomyelia, and painful scar; specific neuralgias at any location of the body; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; and connective tissue disorders.

17) A method for treating a subject suffering fro neuropathy, said method comprising topically administering to the subject an effective amount of ketamine formulated in a pharmaceutical topical vehicle that limits systemic penetration of ketamine.

18) The treatment of claim 17, wherein the topical vehicle is designed to maximize ketamine delivery through the stratum corneum and into the epidermis or dermis or dermatome.

19) The treatment of claim 17, wherein the transdermal vehicle is a skin retardant.

20) The treatment of claim 17, wherein the neuropathy is selected from peripheral neuropathy, phantom limb pain, reflex-sympathetic dystrophy, causalgia, syringomyelia, and painful scar; specific neuralgias at any location of the body; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; AIDS-related pain syndromes; and connective tissue disorders.
